SEQUENCE LISTING (12 sequences)

SEQ ID NO: 1 - DNA, 18 bp, Artificial Sequence
  Synthetic Primer
ttctcttggctgttactg

SEQ ID NO: 2 - DNA, 18 bp, Artificial Sequence
  Synthetic Primer
ttctgtcactctcctctt

SEQ ID NO: 3 - DNA, 19 bp, Artificial Sequence
  Synthetic Primer
aagtaacagcaatgaagtg

SEQ ID NO: 4 - DNA, 18 bp, Artificial Sequence
  Synthetic Primer
ccagttcctcacattctt

SEQ ID NO: 5 - DNA, 18 bp, Artificial Sequence
  Synthetic Primer
gtggagcaggtgaagaat

SEQ ID NO: 6 - DNA, 23 bp, Artificial Sequence
  Synthetic Primer
tctatgtagttgatgaagatgtc

SEQ ID NO: 7 - DNA, 21 bp, Artificial Sequence
  Synthetic Primer
ctctggtaaagtggatattgt

SEQ ID NO: 8 - DNA, 20 bp, Artificial Sequence
  Synthetic Primer
ggtggaatcatattggaaca

SEQ ID NO: 9 - DNA, 18 bp, Artificial Sequence
  Synthetic Primer
ttaactcaagtggcatag

SEQ ID NO: 10 - DNA, 18 bp, Artificial Sequence
  Synthetic Primer
tgattcaatgacgcttat

SEQ ID NO: 11 - DNA, 20 bp, Artificial Sequence
  Synthetic Primer
aaggctgtgggcaaggtcat

SEQ ID NO: 12 - DNA, 20 bp, Artificial Sequence
  Synthetic Primer
cgtcagatccacgacggaca

(19) United States
(12) Patent Application Publication
(10) Pub. No.: US 2021/0205366 A1
(43) Pub. Date: Jul. 8, 2021

(54) METHOD FOR ACTIVATING IMMUNOCYTES IN VITRO

(71) Applicant: WUHAN BINHUI BIOTECH CO.,LTD., WUHAN (CN)

(72) Inventors: BINLEI LIU, WUHAN (CN); YANG WANG, WUHAN (CN); HAN HU, WUHAN (CN); JING JIN, WUHAN (CN); RUNYANG WANG, WUHAN (CN)

(21) Appl. No.: 17/134,209

(22) Filed: Dec. 25, 2020

Related U.S. Application Data
(63) Continuation of application No. PCT/CN2019/093028, filed on Jun. 26, 2019.

(30) Foreign Application Priority Data
Jun. 28, 2018 (CN) 201810706038.9

(51) Int. Cl. A61K 35/17; C12N 7/04

(52) U.S. Cl. CPC A61K 35/17; C12N 7/04

(57) ABSTRACT

The present disclosure a method for activating immunocytes in vitro by VAK technique. Autologous immunocytes of the cancer patient can be activated in vitro by the VAK technique, and by re-infusing these activated immunocytes into the patient's body, a good anti-tumor effect can be achieved, and because these immunocytes are autologous immunocytes, there is no rejection, being safe and reliable.

### CROSS-REFERENCE TO RELATED APPLICATIONS

The present application is a continuation-application of International Patent Application (PCT) No. PCT/CN2019/093028 filed on Jun. 26, 2019, which claims foreign priority of Chinese Patent Application No. 201810706038.9, filed on Jun. 28, 2018, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Mar. 26, 2021, is named SY1US200186_SL.txt and is 2,595 bytes in size.

### FIELD

The present disclosure relates to a method for activating immunocytes in vitro by VAK technique, and belongs to the field of biotechnology.

### BACKGROUND

In the prior art, multiple methods have been developed for cancer therapy, such as surgical excision, radiotherapy, chemotherapy, anticancer drugs, intratumoral injection of virus and the like, but because the cancer patients have low autoimmune function or are tolerant to cancer cells, these methods sometimes have poor effects. Therefore, during the cancer therapy, enhancing patient's own immunity and activating immunological reaction against the cancer cells are very critical.

### SUMMARY

The purpose of the present disclosure is to provide a method for activating immunocytes in vitro by VAK technique, and cancer cells can be killed and meanwhile the release of tumor associated antigen is promoted, being beneficial to inducing a specific anti-tumor immunological reaction.

In order to realize the above purpose, the method for activating immunocytes in vitro by VAK technique provided by the present disclosure, comprising the following steps:

(1) Isolating immunocytes from peripheral blood or malignant pleural effusion samples of a patient;

(2) Co-incubating inactivated herpes simplex viruses with the immunocytes, to activate the immunocytes; and

(3) Removing the inactivated herpes simplex viruses to obtain the activated immunocytes.

Preferably, said step (3) is washing with a phosphate buffer to remove the viruses, to obtain the activated immunocytes.

Optionally, said body fluid containing the immunocytes is peripheral blood or malignant pleural effusion.

Optionally, said viruses are DNA or RNA viruses, including but not limited to the herpes simplex viruses.

Optionally, said viruses are recombinant type herpes simplex viruses or wild-type herpes simplex viruses.

Optionally, said viruses are herpes simplex viruses type I or herpes simplex viruses type II.

Preferably, said viruses are recombinant herpes simplex viruses type II, with a preservation number of CGMCC No. 3600.

The method provided by the present disclosure adopts the VAK (Virus activated killer) technique, which is the inventors' innovative technique, and the virus-activated immunocytes are re-infused into the body of the patient, thereby achieving the anti-tumor effect. Some cancer patients have low immunocytes function or are tolerant to the cancer cells, the virus can effectively activate these immunocytes (the immunocytes activated by the VAK technique are called VAK cells for short), such that the immunocytes achieve a powerful tumor killing effect, and VAK cells kill the cancer cells and meanwhile promote release of tumor associated antigen, being beneficial to inducing a specific anti-tumor immunological reaction.

The method provided by the present disclosure has the beneficial effects: autologous immunocytes of the cancer patient can be activated in vitro by the VAK technique, and by re-infusing these activated immunocytes into the patient's body, a good anti-tumor effect can be achieved, and because these immunocytes are autologous immunocytes, there is no rejection, being safe and reliable.

### BRIEF DESCRIPTION OF THE DRAWINGS

FIG. 1 shows a timeline of a mouse experiment scheme.

FIG. 2 shows the content of CD4⁺ T cells in peripheral blood of oHSV2 single stimulated mouse; in the figure, (A) is a group in which the mouse is not implanted with tumor and only a formulation buffer solution is injected; (B) is an experiment group in which the mouse is not implanted with tumor and only oHSV2 is injected; (C) is a group in which the mouse is implanted with tumor and the formulation buffer solution is injected; (D) is an experiment group in which the mouse is implanted with tumor and the oHSV2 is injected.

FIG. 3 shows the content of CD4⁺T cells in peripheral blood of oHSV2 multiple stimulated mouse; in the figure, (A) is a group in which the mouse is not implanted with tumor and only the formulation buffer solution is injected; (B) is an experiment group in which the mouse is not implanted with tumor and only the oHSV2 is injected; (C) is a group in which the mouse is implanted with tumor and the formulation buffer solution is injected; (D) is an experiment group in which the mouse is implanted with tumor and the oHSV2 is injected.

FIG. 4 shows the content of CD4⁺ T cells in spleen of oHSV2 multiple stimulated mouse; (A) is a group in which the mouse is not implanted with tumor and only the formulation buffer solution is injected; (B) is an experiment group in which the mouse is not implanted with tumor and only the oHSV2 is injected; (C) is a group in which the mouse is implanted with tumor and the formulation buffer solution is injected; (D) is an experiment group in which the mouse is implanted with tumor and the oHSV2 is injected.

FIG. 5 shows change in the expression level of mouse IFN-γ cytokines at different time points.

FIG. 6 shows that oHSV2 can promote proliferation of human PBMC.

FIG. 7 shows the DAPI staining result in the PBMC group.

FIG. 8 shows the DAPI staining result of stimulating the proliferation of PBMC by oncolytic virus.

FIG. 9 shows the content of cytokine at 48 h (Donor: LXX).

FIG. 10 shows change in the expression level of IFN-γ cytokine at different time points (Donor: JJ).

FIG. 11 shows the dynamic change in IFN-γ in VAK at different time points (Donor: JJ).

FIG. 12 shows a cell counting diagram of UV-oHSV2 stimulated mouse PBMC proliferation. Wherein, A, B and C are respectively counting diagrams of PBMC proliferation at 0, 24 hours and 48 hours.

FIG. 13 shows an effect comparison diagram of in-vitro killing CT26 by UV-oHSV2 stimulated mouse PBMC (all experiment groups).

FIG. 14 shows an effect comparison diagram of in-vitro killing CT26 by UV-oHSV2 stimulated mouse PBMC (not containing PLL).

FIG. 15 shows an effect comparison diagram of in-vitro killing 4T1 by UV-oHSV2 stimulated mouse PBMC (all experiment groups).

FIG. 16 shows an effect comparison diagram of in-vitro killing 4T1 by UV-oHSV2 stimulated mouse PBMC (not containing PLL).

FIG. 17 shows a mouse tumor size increase trend chart of in-vivo killing CT26 by UV-oHSV2 stimulated mouse PBMC (on day 17).

FIG. 18 shows a mouse tumor size increase trend chart of the UV-oHSV2 stimulated mouse PBMC group after reinjection of CT26 (on day 40).

FIG. 19 shows a mouse tumor size increase trend chart of in-vivo killing 4T1 by UV-oHSV2 stimulated mouse PBMC (at 40 day).

FIG. 20 shows a cell counting diagram of the UV-oHSV2 stimulated human PBMC proliferation. Wherein, A, B and C respectively shows counting diagrams of PBMC proliferation at 24 hours, 48 hours and 72 hours.

FIG. 21 shows an effect comparison diagram of in-vitro killing LoVo by PBMC cells of volunteer LBL (all experiment groups).

FIG. 22 shows an effect comparison diagram of in-vitro killing LoVo by PBMC cells of volunteer LBL (not containing PLL).

FIG. 23 shows an effect comparison diagram of in-vitro killing LoVo by PBMC cells of the volunteer GQX (all experiment groups).

FIG. 24 shows an effect comparison diagram of in-vitro killing LoVo by PBMC cells of the volunteer GQX (not containing PLL).

FIG. 25 shows an effect comparison diagram of in-vitro killing LoVo by PBMC cells of the volunteer ST (all experiment groups).

FIG. 26 shows an effect comparison diagram of in-vitro killing LoVo by PBMC cells of the volunteer SXT (all experiment groups).

FIG. 27 shows an effect comparison diagram of in-vitro killing BGC823 by PBMC cells of the volunteer GQX (all experiment groups).

FIG. 28 shows an effect comparison diagram of in-vitro killing BGC823 by PBMC cells of the volunteer KZH (all experiment groups).

FIG. 29 shows an effect comparison diagram of in-vitro killing BGC823 by PBMC cells of the volunteer LBL (all experiment groups).

FIG. 30 shows an effect comparison diagram of in-vitro killing BGC823 by PBMC cells of the volunteer SXT (all experiment groups).

FIG. 31 shows an effect comparison diagram of in-vivo killing of LoVo by the UV-oHSV2 stimulated human PBMC (all experiment groups).

FIG. 32 shows an effect comparison diagram of the in-vitro killing LoVo in different groups of the volunteer FZY.

FIG. 33 shows a flow cytometry diagram of NK cells under activated state in the human VAK.

FIG. 34 shows a flow cytometry diagram of CD4⁺T cells under activated state in the human VAK.

FIG. 35 shows a flow cytometry diagram of CD8⁺T cells under activated state in the human VAK.

FIG. 36 shows a flow detection diagram of killing LOVO by activated NK in VAK.

### DETAILED DESCRIPTION OF THE EMBODIMENTS

The disclosure is further described in detail below, in conjunction with specific examples and accompanying drawings.

The virus involved in the experiment of this example is herpes simplex virus, and was inactivated. Such herpes simplex virus includes recombinant herpes simplex virus type I, recombinant herpes simplex virus type II, wild-type herpes simplex virus type I, and wild-type herpes simplex virus type II, as follows:

oHSV1: recombinant herpes simplex virus type I, its microbial preservation number is CGMCC No. 6397, and ICP34.5 gene and ICP47 gene are knocked out in the herpes simplex virus (disclosed in authorized China patent application CN201210337627.7 “Recombinant herpes simplex virus, preparation method and application thereof”)

oHSV2: recombinant herpes simplex virus type II, its preservation number is CGMCC No. 3600. For the preserved biomaterial H2d3d4-hGF strain, the meaning of its strain number is as follows: H2 refers to herpes simplex virus type II HG52 strain (HSV2); d3 refers to a strain in which ICP34.5 is knocked out; d4 refers to a strain in which ICP47 is knocked out; hGF refers to an expression cassette in which human granulocyte-macrophage colony-stimulating factor (hGM-CSF) is inserted (disclosed in authorized China patent application CN201010116275.3 “Vector of recombinant herpes simplex virus type II and preparation method, recombinant virus, pharmaceutical composition and application thereof”).

HSV1: wild-type herpes simplex virus type I, Catalogue No. 0104151v; the virus is purchased from UK National Collection of Pathogenic Viruses (NCPV).

HSV2: wild-type herpes simplex virus type II, Catalogue No. 0104152v; the virus is purchased from UK National Collection of Pathogenic Viruses (NCPV).

Part I: Preparation of Test

### Test Example 1. Design of Mouse Experiment Method

1.1 Activation of the Proliferation of Mouse Immunocytes by Oncolytic Virus

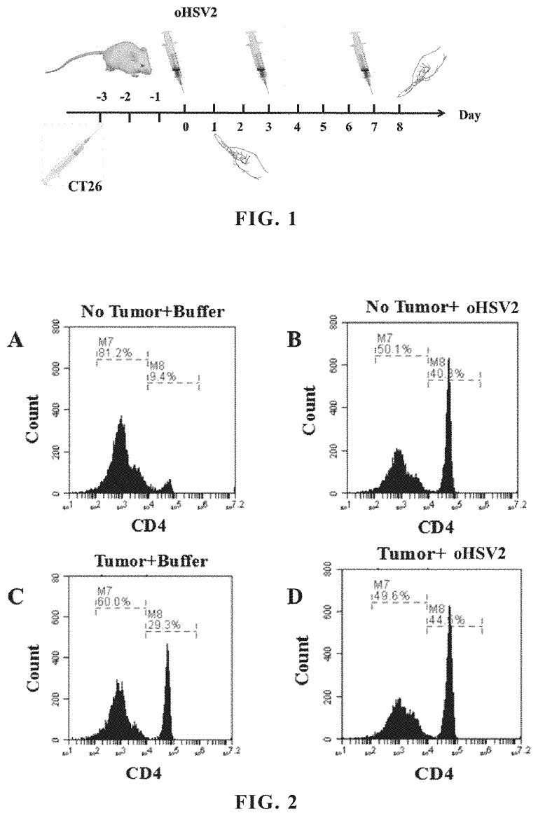

The mouse is of a BLBA/c strain, the tumor model of the mouse is CT26 (the tumor cell line of mouse colon cancer), and the number of the implanted tumors in the mouse is 2×10⁵ cells/mouse. The tested mice were grouped into four groups with 4 mice in each of the groups, which were a tumor implantation group (without treatment), a tumor implantation+buffer solution group, a tumor implantation+oHSV2 group and an oHSV2 group (without tumor implantation). The tumor implantation group (without treatment) only serves as a control group, and any subsequent operation procedures were not conducted after completion of the tumor implantation. In the tumor implantation+buffer solution control group, a buffer solution containing oHSV2 virus was injected for three times in succession. In the tumor implantation+oHSV2 experiment group, in the subsequent experiment, the tumor implantation operations were conducted for three times in succession. In the experiment group in which only oHSV2 was injected, the tumor implantation was not conducted, only oncolytic viruses were injected for three times in succession. The timeline of the experiment in which the tumor was implanted and the oncolytic viruses were injected was seen in FIG. 1. Three days after the mice tumor implantation, observation was conducted, when the tumor size reached 100 mm³, subcutaneous injection in situ may be conducted for three times in succession. On the next day after third injection treatment on the mice, samples were collected from all the mice.

### Test Example 2. Preparation of Mouse VAK

2.1 Collection of Peripheral Blood: The Mouse was Taken Out, and Blood was Collected from the Eyeball.

2.2. Mouse PBMC was isolated from the mouse blood by using a commercially available kit (a mouse peripheral blood lymphoid isolation kit from TianJin HaoYang Biological Manufacture Co., Ltd).

2.3. Preparation of Mouse VAK

1) The mouse blood in a heparin sodium anticoagulation tube was put into a clean centrifugal tube, an equal volume of mouse blood sample diluent was added, and the mixture was slowly blown and beaten to be even, ready for use.

2) To a lymphocyte isolation tube of 15 mL, an equal blood volume of mouse peripheral blood lymphocyte isolation medium was added, letting the lymphocyte isolation medium fall into the bottom of a partition plate, and if the lymphocyte isolation medium could not fall into the bottom, centrifugal operation was conducted. The diluted blood sample was slowly added along the tube wall, and centrifuged by using a medical centrifugal machine 820 at 20° C. for 25 minutes.

3) An uppermost serum layer was taken out and put in a clean centrifugal tube of 15 mL, placed in a water bath at 56° C., inactivated for 30 minutes, and centrifuged under 3500 rpm at 20° C. for 10 minutes, the precipitate was discarded, and the supernatant was left, ready for use.

4) A middle milk-white lymphocyte layer was put in a clean centrifugal tube of 15 mL, and an equal volume of PBS was added, and mixed well with the lymphocyte layer, the mixture was centrifuged under 820 g at 20° C. for 10 minutes, the supernatant was discarded, and the precipitate was left.

5) The cell precipitate obtained in 4) was washed, centrifuged under 820 g at 20° C. for 10 minutes, the supernatant was discarded, and the precipitate was left. The operation was repeatedly conducted for one time.

6) The finally obtained precipitate was re-suspended with 4 mL of serum-free medium.

7) oHSV2 was sampled and put in an EP tube, and processed for 30 minutes by ultraviolet irradiation for inactivation, ready for use.

8) 100 μL of cell suspension was added in 900 μL of PBS, and counted by using a blood counting chamber.

9) The collected cells were co-incubated with oHSV2 with MOI=1, 0.1 and 0.01. The set groups are respectively a blank control group (PBMC), a solvent control group (PBMC and a stabilizer), a positive control group (PBMC and PHA) as well as stimulation groups with MOI=1, 0.1 and 0.01, totally six groups.

10) A pre-mixed suspension was placed in a six-well plate, and incubated in a CO₂ incubator.

### Test Example 3. Preparation of Mouse Spleen Single Cell Suspension

3.1. Organ Dissection

After the mouse was killed by cervical dislocation, and the thoracic cavity of the mouse was cut open by a surgical scissor. The entire spleen was taken out, and a portion of spleen tissue was cut off

3.2. Grinding of Spleen

1) The spleen was put in a 1.5 mL EP tube, and an appropriate amount of PBS (1×) was added, and temporarily stored.

2) A mortar, filter cloth and an injector plug were washed twice with PBS. The mortar mouth was covered by the filter cloth, the filter cloth was fixed by the thumb and middle finger of the left hand, a tumor body was put in the middle, the spleen was cut into pieces by a scissor, and the filter cloth was fixed. The spleen was ground with the injector plug of 5 mL, during the grinding, the filter cloth was pressed, and the cells were ground out by resilience of the filter cloth. After the spleen was ground for some time, 1 mL of PBS was added, finally, during the grinding, 3 mL of PBS (1×) was added, a white connective tissue was left on the filter cloth, and grinding liquid was mixed well with 1 mL of tip to obtain 3 mL of cell suspension, and 10 μL of suspension was sucked.

3) 2 mL of the suspension was sucked and put in a centrifugal tube of 15 mL, and PBS (1×) was added in a volume ratio of 1:4, i.e., 8 mL, and mixed well with the suspension by 1 mL of the tip, the mixture was allowed to stand for 10 minutes, the supernatant was discarded, 5 mL of PBS was added and uniform mixing was conducted by 1 mL of the tip.

4) The mixture was centrifuged under 820 g at 20° C. for 5 minutes. A cell precipitate was obtained, and re-suspended with 3 mL of PBS.

5) 1 mL of cell suspension was sucked, and filtered with filter cloth of 300-mesh, and the filtrate was put in a 15 mL centrifugal tube. After uniform mixing, 200 μL of sample was diluted by 1-fold, and then counted by a flow cytometer.

6) The cells were diluted, and the cell number was required at 3000-4000 cells/4.

7) The diluted cells were sub-packed into an EP tube of 200 μL, and the cell stock solution was mixed well by a vortex mixer, to obtain a single cell suspension of the spleen.

### Test Example 4. Preparation of Human VAK

When the blood was collected, the volunteer was required to be in good health and in normal condition, without inflammation. One day before the blood collection, the volunteer was required to eat a light diet, without drinking wine, and to ensure sufficient sleep. At the time of blood collection in the morning of next day, the stomach was empty.

4.1. Processing of Blood Sample

1) Human PBMC was isolated from human blood by using a commercially available kit (Human peripheral blood lymphoid isolation kit from TianJin HaoYang Biological Manufacture Co., Ltd).

2) The finally obtained PBMC cell precipitate was re-suspended by 4 mL of serum-free medium DMEM/F12.

3) oHSV2 was sampled and put into an EP tube, processed for 30 minutes by ultraviolet radiation, and inactivated, ready for use.

4) 100 μL of cell suspension was added in 900 μL of PBS, and counted by a blood counting chamber.

5) The collected cells were co-incubated with oHSV2 with MOI=1, 0.1 and 0.01. The set groups are respectively a blank group control group (PBMC), a solvent control group (PBMC and a stabilizer), a positive control group (PBMC and PHA) and stimulation groups with MOI=1, 0.1 and 0.01, totally six groups.

6) A pre-mixed suspension was placed into a six-well plate, and incubated in a CO₂ incubator.

4.2. Related Information of VAK Volunteers

Related information of the volunteers for VAK preparation is seen in the following Table 1.

TABLE 1
Related information of the volunteer for blood collection

| Donor | Gender | Age |
|---|---|---|
| LBL | Man | 55 |
| LXX | Woman | 37 |
| LHP | Woman | 42 |
| CLK | Man | 25 |
| JJ | Woman | 25 |
| WRY | Man | 23 |
| GQX | Man | 40 |
| SXT | Man | 30 |
| KZH | Man | 26 |
| ST | Man | 45 |

A grouping experiment was conducted as follows for the above-described volunteers, each group was divided into 6 items, including a PBMC control group, a buffer control group, and three groups with decreased virus addition volume, and a PHA control group, the following Table 2 is a grouping experiment table for a volunteer LBL, the grouping experiment table of other volunteers is similar to this table, only the addition amount of the reagent and virus were adjusted in proportion according to the difference in number of the volunteer cell suspension.

TABLE 2
Related parameters in VAK preparation (Donor: LBL)

| | | | MOI = | MOI = | MOI = | |
|---|---|---|---|---|---|---|
| Groups | PBMC | Buffer | 1 | 0.1 | 0.01 | PHA |
| Volume of | 500 | 500 | 500 | 500 | 500 | 500 |
| cell | μL | μL | μL | μL | μL | μL |
| suspension | | | | | | |
| Volume of | 250 | 250 | 250 | 250 | 250 | 250 |
| autologous | μL | μL | μL | μL | μL | μL |
| plasma | | | | | | |
| 100 × | 25 | 25 | 25 | 25 | 25 | 25 |
| diabody | μL | μL | μL | μL | μL | μL |
| Volume of | 0 | 360 | 0 | 0 | 0 | 0 |
| buffer | | μL | | | | |
| Volume of | 0 | 0 | 360 | 36 | 3.6 | 0 |
| virus | | | μL | μL | μL | |
| PHA | 0 | 0 | 0 | 0 | 0 | 7.5 |
| | | | | | | μL |
| Serum-free | 1725 | 1365 | 1365 | 1690 | 1725 | 1720 |
| medium | μL | μL | μL | μL | μL | μL |

### Test Example 5. Detection of Cytokines by qPCR Method

5.1 Primer sequence

| Human IFN-γ: | Human IL-15 |
|---|---|
| F: TTCTCTTGGCTGTTACTG | F: AAGTAACAGCAATGAAGTG |
| (SEQ ID NO: 1) | (SEQ ID NO: 3) |
| R: TTCTGTCACTCTCCTCTT | R: CCAGTTCCTCACATTCTT |
| (SEQ ID NO: 2) | (SEQ ID NO: 4) |
| Human IL-10 | Human GAPDH |
| F: GTGGAGCAGGTGAAGAAT | F: CTCTGGTAAAGTGGATATTGT |
| (SEQ ID NO: 5) | (SEQ ID NO: 7) |
| R: TCTATGTAGTTGATGAAG | R: GGTGGAATCATATTGGAACA |
| ATGTC | (SEQ ID NO: 8) |
| (SEQ ID NO: 6) | |
| Mouse IFN-γ | Mouse GAPDH |
| F: TTAACTCAAGTGGCATAG | F: AAGGCTGTGGGCAAGGTCAT |
| (SEQ ID NO: 9) | (SEQ ID NO: 11) |
| R: TGATTCAATGACGCTTAT | R: CGTCAGATCCACGACGGACA |
| (SEQ ID NO: 10) | (SEQ ID NO: 12) |

5.2 qPCR

5.2.1 Preparation Reaction System

| Component | Volume |
|---|---|
| RNase-free ddH₂O | 2 μL |
| SYBR green Mix | 5 μL |
| upstream primer | 1 μL |
| downstream primer | 1 μL |
| template | 1 μL |

5.2.2 PCR Amplification Reaction Condition

3 parallel holes were set for each reaction, and the qPCR reaction was conducted according to the following operation procedure:

| Cycle number | Temperature | Time |
|---|---|---|
| 1 | 95° C. | 20 s |
| 49 | 95° C. | 1 s |
| | 60° C. | 20 s |
| 1 | 60° C. | 5 s |
| 1 | 95° C. | 1 s |

The primers and conditions used in the above-described experiments are all from the disclosed literatures.

Part II. Experiment, Results and Discussion

### Experimental Example 1. oHSV2 can Promote Proliferation of CD4⁺T Cells in the Mouse Peripheral Blood

1.1) Proliferation of CD4⁺T Cells in the oHSV2 Single Stimulated Mouse Peripheral Blood

The animal experiment of the mouse was conducted according to the scheme design of Test Example 1. On the day of the tumor implantation in the mouse, viruses and a formulation buffer solution were injected, on the next day i.e., (the first day), the mouse peripheral blood was collected and PBMC was isolated according to the method in Test Example 2. After the peripheral blood was processed with a lymphocyte isolation medium, the content of CD4⁺T cells in the mouse peripheral blood was detected by flow cytometry. As shown in FIG. 2A, in the control group in which the mouse was not implanted with tumor cells, and only the formulation buffer solution was injected, the content of CD4⁺T cells was only 9.4%; as shown in FIG. 2B, in the experimental group in which tumor implantation was not conducted, only under the stimulation condition of oHSV2, the content of CD4⁺T cells was 40.3%; as shown in FIG. 2C, in the experimental group in which the mouse was implanted with tumor cells and only the formulation buffer solution was injected, the content of CD4⁺T cells was 29.3%. In the experimental results as shown in FIG. 2D, the mouse was implanted with tumor and oHSV2 was injected, and the content of CD4⁺T cells was 44.5%.

1.2) Proliferation of CD4⁺T in oHSV2 Multiple Stimulated Mouse Peripheral Blood

The experimental scheme of the mouse was conducted according to Test Example 1. On the day of tumor implantation in the mouse, first injecting treatment was conducted, on the next day after the third injection treatment (i.e., 8^{th} day) a according to the experimental method of Test Example 2, blood was collected from the eyeball of the mouse. On the 8th day, the mouse was processed, after the peripheral blood was processed with the lymphocyte isolation medium, change in the content of CD4⁺T cells in the mouse peripheral blood was detected by the flow cytometry. As shown in FIG. 3, in the group in which tumor was not implanted and the formulation buffer solution was injected, the content of CD4⁺T cells was 3.5%, in the group in which the tumor was implanted and inactivated oHSV2 was injected, the content of CD4⁺T cells was 16.7%, in the group in which the tumor was implanted and the formulation buffer solution was injected, the content of CD4⁺T cells was 13.8%. But in the experiment group in which the tumor was implanted and inactivated oHSV2 was injected, the content of CD4⁺T cells was 35.7%. For the mouse under a tumor implantation state, due to its own immunological reaction effect against the tumor cell, the CD4⁺T cells within its body was increased. Meanwhile on this basis, due to injection of the inactivated oncolytic virus, percentage content increase of the CD4⁺T cells could be motivated more strongly.

The Experimental result under this condition suggests that inactivated oHSV2 can activate proliferation of the CD4T cells in the body of the mouse.

### Experimental Example 2. Change of CD4⁺T Cell Content in Mouse Spleen

The experimental scheme of the mouse was conducted according to Test Example 1. On the day of tumor implantation in the mouse, first injection treatment was conducted, on the next day after third injection treatment (i.e., 8^{th} day), according to the method in Test Example 2, the mouse was dissected and the spleen was isolated.

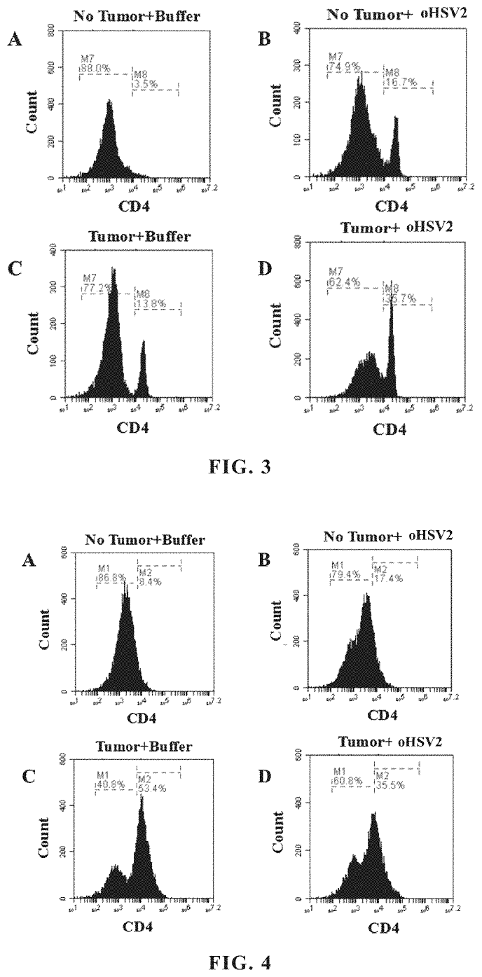

The content of CD4⁺T cells in mouse spleen was detected by flow cytometry. As shown in FIG. 4, the experimental result indicated that, in the control group in which the tumor was not implanted and only the formulation buffer solution was injected, the positive rate was 8.4%, in the experimental group in which the tumor was not implanted and only ultraviolet inactivated oHSV2 was injected, the content of CD4⁺T cells was 17.4%, in the group in which the tumor was implanted and only the formulation buffer solution was injected, the content of CD4⁺ cells was 53.4%, and in the group in which the tumor was implanted and oHSV2 was injected, the content of CD4⁺ cells was 35.5%. Compared with the group in which tumor was not implanted and a stabilizer was added, the CD4⁺ cell contents in other experiment groups were all increased.

### Experimental Example 3. oHSV2 can Promote IFN-γ Release in Mouse PBMC

According to the method of Test Example 2, collection of mouse peripheral blood, isolation of mouse PBMC, and preparation of mouse VAK were conducted in sequence. cDNA of the mouse was extracted by the existing method, and qPCR was conducted according to Test Example 5, and the expression level of IFN-γ was detected.

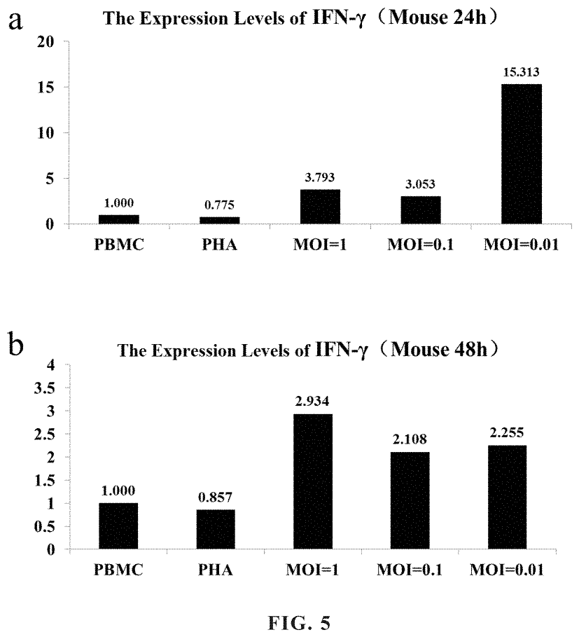

In this experiment, 20 BALB/c male mice aged 7 weeks were used. The detection times were 24 hours and 48 hours. The expression levels of IFN-γ at different time points are as shown in the following FIG. 5.

After preparation of the mouse VAK, as shown in a in FIG. 5, when the incubation time was 24 h, and the mutiplicity of infection (MOI) was 1, 0.1 and 0.01, compared with that of the blank PBMC group, the magnifications of the relative expression level of mRNA are respectively 3.793, 3.053 and 15.313-fold. Under this state, the inactivated oHSV2 can stimulate the transcriptional expression of IFN-γ of single karyoblast in the mouse peripheral blood, particularly when the mutiplicity of infection (MOI) was 0.01, the magnification was up to 15-fold. When the incubation time was 48 hours, and the mutiplicity of infection (MOI) was 1, 0.1 and 0.01, compared with that of the PBMC group, the magnifications of the expression level are respectively 2.934, 2.108 and 2.255-fold.

### Experimental Example 4. oHSV2 can Promote Proliferation of Human Peripheral Blood PBMC

4.1) Result of Cell Counting Experiment

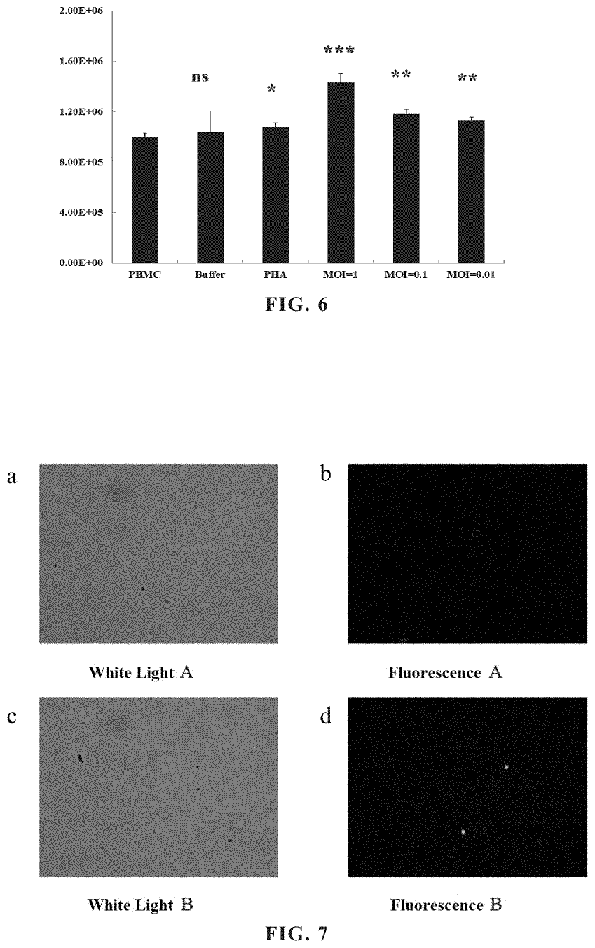

VAK was prepared according to the method in Test Example 4, and put into a carbon-dioxide incubator for culture. After the ultraviolet inactivated oncolytic virus (oHSV2) was co-incubated with PBMC for 72 h, cell counting was conducted. The experimental results are as shown in the following FIG. 6, and six groups were set in the experiment, and were respectively a PBMC control group, a formulation buffer solution group, a positive stimulator group (a PBMC group), and three experiment groups with different mutiplicity of infections (MOI). After the statistical analysis on the subsequent five groups and the PBMC group, there is no difference in the Buffer group; and the positive stimulator group has a statistical difference; the group with MOI=1 has a very significant statistical difference; the two groups with MOI=0.1 and MOI=0.01 have significant statistical difference. It can be seen from this that under the stimulation effect of the oncolytic virus (oHSV2), proliferation of human PBMC can be effectively promoted.

4.2) Result of DAPI Staining Experiment

In order to further explore the proliferation status under the conditions that the MOI of oHSV2 was 0.01, and virus was co-incubated with the cell for 48 h, photos were taken and observed by an inverted fluorescence microscope. 15 μL of prepared VAK was placed onto a clean slide, and covered with a coverslip.

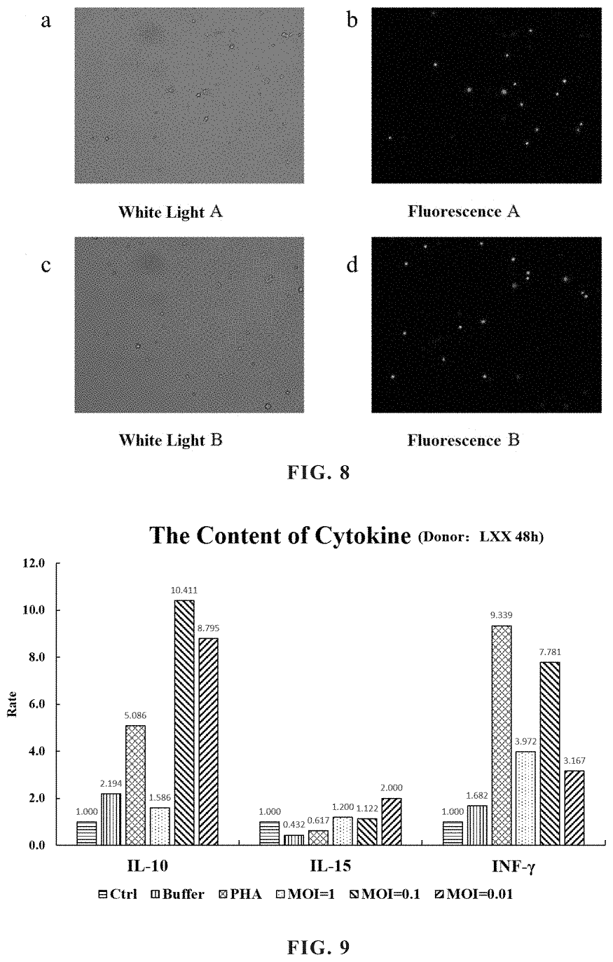

The exposure condition set for the inverted fluorescence microscope is IS. The fluorescent images of white light and blue light are in the same field of view. A and B are the observation results of the same sample in different fields of view. In the blank control group, as shown in FIG. 7, blue fluorescence represents peripheral blood mononuclear cells stained by DAPI dye. Under the condition of fluorescent observation, in the negative control group (only containing PBMC, not containing the stimulator), under the condition of white light photo-taking, the cell distribution is sparse, the cell morphology is small, whereas in the corresponding field of view, the number of the blue fluorescent bright spots is small, the bright spot distribution is sparse, the fluorescence intensity is weak. Under the condition that mutiplicity of infection (MOI) of oHSV2 was 0.01, as show in FIG. 8, under the condition of white light photo-taking, the cell distribution is relatively dense, the cell number is relatively large, the number of the corresponding blue fluorescent bright spot is large, fluorescence intensity is high, significantly stronger than the number of the peripheral blood mononuclear cells under the condition of the blank control group.

Under an ex-vivo condition, by the stimulation effect of oHSV2, PBMC can effectively proliferate. By using a fluorescence microscope, increase in PBMC number can be directly observed, providing an intuitive visual effect. A conclusion is obtained that, after the ex-vivo PBMC was co-incubated with oHSV2 for 72 hours, PBMC can have obvious proliferation.

### Experimental Example 5. oHSV2 can Promote Release of IFN-γ in Human Peripheral Blood PBMC

5.1 Change in Expression Level of Cytokines in the PBMC Under the oHSV2 Stimulation Condition

48 hours after the VAK preparation, the relative expression level of the immunosuppressive regulatory factor IL-10, as well as relative expression levels of positive immune regulatory factors IL-15 and IFN-γ were detected by the qPCR method.

VAK was prepared according to the method in Test Example 4.

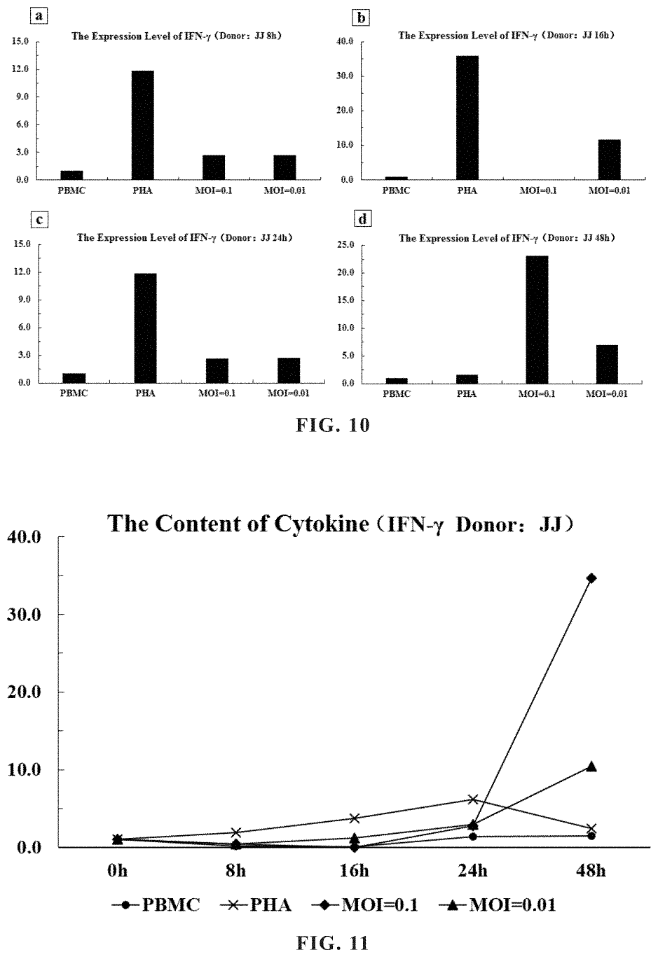

The relative expression level of mRNA in the cells is shown in Table 3 and FIG. 9, wherein the relative expression level of IL-10 presented a state of increase. The change in the expression level of IL-15 was not obvious. The relative expression level of IFN-γ was also obviously increased. The experimental results showed that, compared with the PBMC control group, for Buffer, PHA, MOI=1, MOI=0.1 and MOI=0.01, the relative expression levels were 1.682, 9.339, 3.972, 7.781 and 3.167 folds. In the results, the positive and negative regulatory factors are both increased, a supposed interpretation for this phenomenon is in that while the cell is rapidly releasing a positive regulatory factor in response to external stimulation, the cell also releases some negative regulatory factors, to prevent a state of unconstrained release of the positive regulatory factors, such that a buffered state is achieved. This supposal still needs a lot of subsequent work for investigation and verification.

TABLE 3
Content of cytokines at 48 h (Donor: LXX)

| 48 h | IL-10 | IL-15 | IFN-γ |
|---|---|---|---|
| Control | 1.000 | 1.000 | 1.000 |
| Buffer | 2.194 | 0.432 | 1.682 |
| PHA | 5.086 | 0.617 | 9.339 |
| MOI = 1 | 1.586 | 1.200 | 3.972 |
| MOI = 0.1 | 10.411 | 1.122 | 7.781 |
| MOI = 0.01 | 8.795 | 2.000 | 3.167 |

The above experimental results proved that, after oHSV2 was incubated with PBMC, the cells can secret a large number of cytokines IFN-γ, to make a response to the stimulation from the external environment. 5.2) Change in IFN-γ expression level under the stimulation condition at different time points
After oHSV2 was incubated with PBMC, secretion of IFN-γ by the cells can be promoted to make a response to external environment. But whether the expression level of its secretion dynamically changes with the time or not is still not clear.
VAK was prepared according to the method in Test Example 4 (Donor: JJ).
Expression levels of IFN-γ at 0 h, 8 h, 16 h, 24 h and 48 h were investigated. As shown in FIG. 10a, at 8 h after the VAK preparation, the relative expression level in the positive stimulator PHA group was 11.85; and the relative expression levels in the groups with MOI=1 and 0.1 were respectively 2.67 and 2.68-fold. As shown in FIG. 10b, at 16 h, the relative expression level in the positive stimulator group was 35.84-fold; when MOI=0.01, the relative expression level was 11.63-fold, they are all significantly increased. As shown in FIG. 10c, at 24 h, the expression level magnification of IFN-γ is still high. As shown in FIG. 10d, at 48 h, the IFN-γ expression levels in the oHSV2 virus infected groups with MOI=0.1 and MOI=0.01 were respectively 23.10 and 6.96, at this time, there is still a high degree of expression.
Based on the experimental results, it was indicated that under the condition of stimulation by the oncolytic virus, release of IFN-γ is effectively promoted.
However, dynamic change of IFN-γ expression level with the time point is still unknown. Next, the expression change with the dynamic change of time was further investigated. As shown in Table 4 and FIG. 11, in the PBMC group, as shown by the black solid dot, with the increase of time, the difference in IFN-γ expression level is not obvious. But in the PHA positive stimulation group shown by a crossed symbol, with the increase of the time points, its expression level firstly presents increase, but then reaches a peak value at 24 h, and then has a trend of decrease. However, in the oHSV2 virus infected group, all present a trend of gradual increase in IFN-γ expression level with time. Particularly when MOI=0.1, compared with the expression level under a resting state, its expression level at 48 h is up to 34.695 folds. When MOI=0.01, its relative expression level is 10.459 folds of that of 0 h. From this, it is concluded that after the oHSV2 virus is co-incubated with PBMC, expression of IFN-γ can be promoted, and was increased with time.

TABLE 4
Dynamic change in IFN-γ in VAK
at different time points (Donor: JJ)

| Groups | 0 h | 8 h | 16 h | 24 h | 48 h |
|---|---|---|---|---|---|
| PBMC | 1.000 | 0.161 | 0.103 | 1.366 | 1.502 |
| PHA | 1.000 | 1.905 | 3.706 | 6.176 | 2.395 |
| MOI = 0.1 | 1.000 | 0.429 | 0.001 | 2.751 | 34.695 |
| MOI = 0.01 | 1.000 | 0.431 | 1.203 | 2.935 | 10.459 |

### Experimental Example 6. Study of Mouse PBMC (Peripheral Blood Mononuclear Cell) Proliferation Experiment

6.1 Blood sample preparation and mouse PBMC isolation were similar to those in Test Example 2. Mouse PBMC planking was similar to that in Test Example 2, the difference is in that, the set groups are respectively a blank control group (PBMC) and a stimulation group with MOI=1, totally two groups.

6.2 Experimental Results

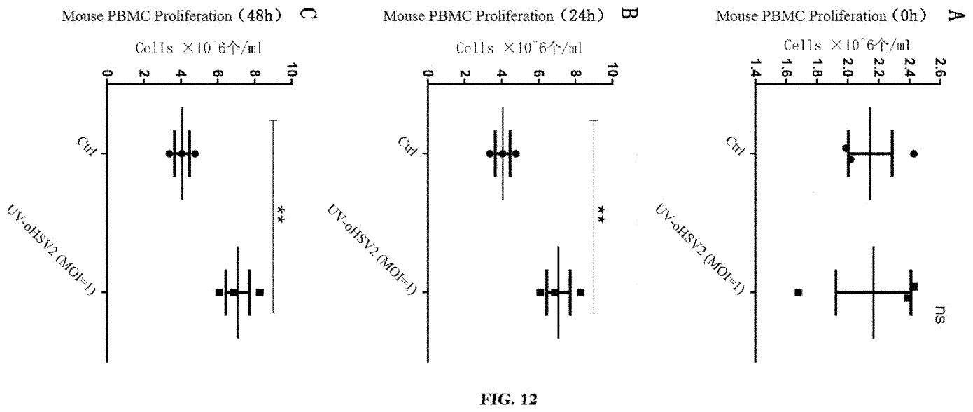

In this experiment, the proliferation is determined on the basis of the result of PBMC counting, if the cell density of PBMC is increased, it is determined that PBMC is proliferated. The above-described planked PBMC were counted at 0 h, 24 h, 48 h, every dot represents one independent experiment, and statistical analysis was conducted on the result obtained in the experiment with GraphPad Prism 6.01 software. t test was adopted for comparison between the groups, the result was analyzed with P<0.05 having a statistical difference. The results are as shown in FIG. 12.

6.3. Discussion on the Results

It is known from the experimental result FIG. 12, at 24 h, there is no significant difference, between the UV-oHSV2 group and the control group, but is can be seen from the trend, the UV-oHSV2 group has a trend of proliferation. At 48 h, the UV-oHSV2 group has a highly significant difference, PBMC is proliferated by 2-3 folds. This suggests that stimulation to mouse PBMC by UV-oHSV2 can make PBMC proliferated, and the degree of activation can be determined according to the subsequent in-vitro and in-vivo killing results. In this UV-oHSV2 stimulated mouse PBMC proliferation experiment, only one result of mutiplicity of infection (MOI) is obtained, i.e., when MOI=1, in the subsequent in-vitro killing, multiple MOI stimulations are increased.

### Experimental Example 7. In-Vitro Killing of CT26 Tumor Cells by UV-oHSV2 Stimulated Mouse PBMC

This experimental example aims to explore whether the PBMC after UV-oHSV2 may be activated or not after 48 h of proliferation, and whether the effect of the PBMC after activation in killing the tumor cells was related to MOI of HSV2 or degree of proliferation or not.

This experiment adopts mouse colon cancer (CT26) cells, and explore whether PBMC after activation through proliferation kills CT26 cells or not by MTT colorimetry.

7.1 Planking of Mouse PBMC was Similar to that in the Test Example 2.

7.2 Preparation of CT26 Cell

The CT26 cells were cultivated according to the conventional method, and the medium was DMEM/F12 containing 10% newborn bovine serum. The cells were digested and collected, and centrifuged under 2800 rpm for 5 minutes, finally the cell precipitate was re-suspended with a DMEM/F12 medium containing 10% newborn bovine serum, the cells were counted, and the cells were diluted to a cell density of 8×10⁵ cell/ml with the DMEM/F12 medium containing 10% newborn bovine serum, ready for use.

7.3 Coating of Polylysine (PLL) Plate (the Method is Conventional Technique in this Field)

7.4 CT26 Cell In-Vitro Killing Planking

The experiment was divided into six groups, and every group has 5 holes, totally 150 μl/well of mixed liquor.

Experiment group one: 50 μl of UV-oHSV2 stimulated PBMC of MOI=0.1, 1+50 μl of CT26+50 μl of DMEM/F12 containing 10% NBS (a well containing PLL).

Experiment group two: 50 μl of UV-oHSV2 stimulated PBMC of MOI=0.1, 1+50 μl of CT26+50 μl of DMEM/F12 containing 10% NBS.

Experiment group three: 50 μl of UV-oHSV2 stimulated PBMC of MOI=0.1, 1+50 μl of CT26+50 μl of DMEM/F12 containing 10% NBS+50 μl of 10-fold diluted pancreatin (pancreatin is used as an adherence inhibitor).

Experiment group four: blank group (containing PBMC) 50 μl+50 μl of CT26+50 μl of DMEM/F12 containing 10% NBS.

Experiment group five: 50 μl of CT26+100 μl of DMEM/F12 containing 10% NBS (a well containing PLL).

Blank group: 150 μl of CT26.

After the planking, a 96-well plate was put in a CO₂ incubator and cultivated for 48˜72 hours.

7.5 MTT Colorimetry Detection (the Detection Method is Conventional Technique in this Field)

7.6 Experimental Result

The absorbance of each well at a wavelength of 492 nm was detected with a multifunctional enzyme marker, the results are as follows:

TABLE 5
Table of the results of killing CT26 in different groups

| | | | | | | Mean |
|---|---|---|---|---|---|---|
| Groups | OD value | | | | | value |
| UV-oHSV2 (PBMC) + | 1.113 | 1.481 | 0.881 | 0.797 | 1.405 | 1.063 |
| PLL MOI = 0.1 | | | | | | |
| UV-oHSV2 (PBMC) + | 0.560 | 0.722 | 0.757 | 0.671 | 0.659 | 0.674 |
| PLL MOI = 1 | | | | | | |
| UV-oHSV2 (PBMC) | 0.966 | 0.920 | 0.890 | 0.869 | 1.026 | 0.934 |
| MOI = 0.1 | | | | | | |
| UV-oHSV2 (PBMC) | 0.718 | 0.634 | 0.683 | 0.696 | 0.628 | 0.672 |
| MOI = 1 | | | | | | |
| UV-oHSV2 (PBMC) + | 0.975 | 0.731 | 0.942 | 0.880 | 0.785 | 0.863 |
| pancreatin MOI = 0.1 | | | | | | |
| UV-oHSV2 (PBMC) + | 0.691 | 0.684 | 0.641 | 0.686 | 1.373 | 0.815 |
| pancreatin MOI = 1 | | | | | | |
| Ctrl (PBMC) | 1.028 | 0.987 | 1.055 | 1.066 | 0.895 | 1.006 |
| Ctrl + PLL | 1.044 | 1.012 | 1.010 | 1.116 | 1.015 | 1.039 |
| Ctrl | 1.081 | 1.334 | 1.226 | 1.062 | 1.019 | 1.144 |

7.7. Discussion on the Results

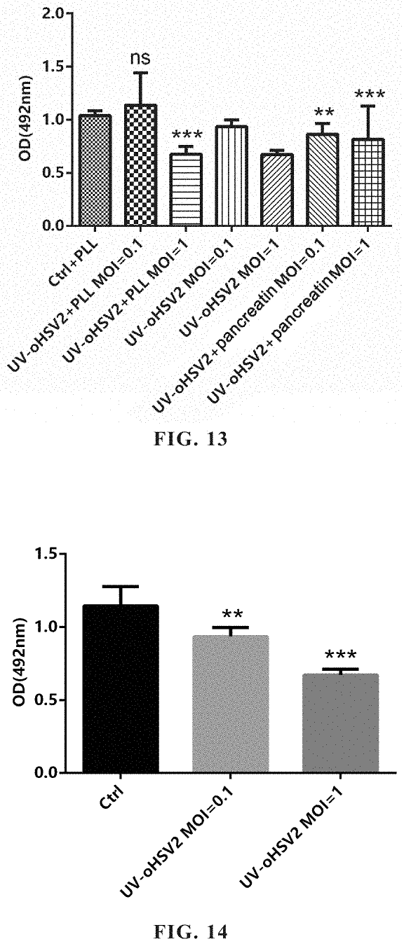

During the experiment, UV-oHSV2 stimulated mouse PBMC was co-incubated with CT26, the multiplicity of infection (MOI) was 3:1, the results are as shown in FIG. 13 and FIG. 14. A group containing polylysine (PLL) and a pancreatin group were set in the experiment, polylysine is a cell adherence promoter, because this experiment adopted co-incubation of PBMC with the tumor cell, to explore of the killing effect of PBMC. In order to eliminate the effect of interrupting adherence of tumor cells by PBMC, a PLL adherence prompter was added. Pancreatin was used as another control: adherence inhibition. It can be found from FIG. 13, when MOI=1, there is a significant difference between the UV-oHSV2+PLL group and the control group, suggesting that PBMC has a killing effect to the CT26 tumor cells, which is not due to the adherence effect of PBMC to CT26 cell infection. It can be seen from FIG. 14, when MOI=0.1 and MOI=1, UV-oHSV2 stimulated mouse PBMC have a strong killing effect on CT26 colon cancers, whereas when high mutiplicity of infection MOI=1, the effect of the UV-oHSV2 stimulated mouse PBMC in killing tumor is better.

### Experimental Example 8. In-Vitro Killing of 4T1 Tumor Cells by UV-oHSV2 Stimulated Mouse PBMC

This experimental example investigated whether PBMC also can kill other tumor cell lines, and here adopted a mouse breast cancer cell line 4T1. The experimental principle adopted an MTT method.

8.1. Planking of Mouse PBMC

Similar to Test Example 2, the difference is in that, the planking groups are respectively untreated PBMC group (Ctrl), MOI=0.1, 10

8.2. The Preparation of 4T1 Cells

The 4TI cells were cultivated according to the conventional method, and the medium was DMEM/F12 containing 10% fetal bovine serum. The cells were digested and collected, and centrifuged under 2800 rpm for 5 minutes, finally the cell precipitate was re-suspended with the DMEM/F12 containing 10% fetal bovine serum, the cells were counted, and the cells were diluted to a cell density of 4×10⁵ cell/ml with the DMEM/F12 containing 10% fetal bovine serum, ready for use.

8.3 Coating of Polylysine (PLL) Plate (the Method is Conventional Technique in this Field)

8.4. 4T1 Cell In-Vitro Killing Planking

The experiment was divided into six groups, and every group has 5 holes, totally 150 μl/well of mixed liquor.

Experiment group one: 50 μl of UV-oHSV2 stimulated PBMC of MOI=0.1, 1+50 μl of 4T1+50 μl of DMEM/F12 containing 10% FBS (a well containing PLL).

Experiment group two: 50 μl of UV-oHSV2 stimulated PBMC of MOI=0.1, 1+50 μl of 4T1+50 μl of DMEM/F12 containing 10% FBS.

Experiment group three: 50 μl of UV-oHSV2 stimulated PBMC of MOI=0.1, 1+50 μl of 4T1+50 μl of DMEM/F12 containing 10% FBS+50 μl of 10-fold diluted pancreatin (pancreatin was used as an adherence inhibitor).

Experiment group four: blank group (containing PBMC) 50 μl+50 μl of 4T1+50 μl of DMEM/F12 containing 10% FBS.

Experiment group five: 50 μl of 4T1+100 μl of DMEM/F12 containing 10% FBS (a well containing PLL).

Blank group: 150 μl of 4T1.

After the planking, the 96-well plate was put in the CO₂ incubator to be cultivated for 48-72 hours.

8.5. MTT Colorimetry Detection (the Detection Method is Conventional Technique in this Field)

8.6. Experimental Results

Absorbance of each well at a wavelength of 492 nm was detected with a multifunctional enzyme marker, the results are as follows:

TABLE 6
Table of results of killing 4T1 in different groups

| | | | | | | Mean |
|---|---|---|---|---|---|---|
| Groups | OD value | | | | | value |
| UV-oHSV2 (PBMC) + | 0.481 | 0.442 | 0.432 | 0.399 | 0.479 | 0.447 |
| PLL MOI = 0.1 | | | | | | |
| UV-oHSV2 (PBMC) + | 0.438 | 0.391 | 0.372 | 0.384 | 0.393 | 0.396 |
| PLL MOI = 1 | | | | | | |
| UV-oHSV2 (PBMC) | 0.396 | 0.404 | 0.500 | 0.659 | 0.443 | 0.480 |
| MOI = 0.1 | | | | | | |
| UV-oHSV2 (PBMC) | 0.376 | 0.376 | 0.379 | 0.362 | 0.391 | 0.377 |
| MOI = 1 | | | | | | |
| UV-oHSV2 (PBMC) + | 0.431 | 0.422 | 0.416 | 0.422 | 0.431 | 0.424 |
| pancreatin MOI = 0.1 | | | | | | |
| UV-oHSV2 (PBMC) + | 0.390 | 0.379 | 0.360 | 0.372 | 0.387 | 0.378 |
| pancreatin MOI = 1 | | | | | | |
| Ctrl (PBMC) | 0.380 | 0.456 | 0.457 | 0.382 | 0.440 | 0.423 |
| Ctrl + PLL | 0.479 | 0.39 | 0.401 | 0.437 | 0.451 | 0.423 |
| Ctrl | 0.408 | 0.416 | 0.404 | 0.437 | 0.451 | 0.423 |

8.7. Discussion on Results

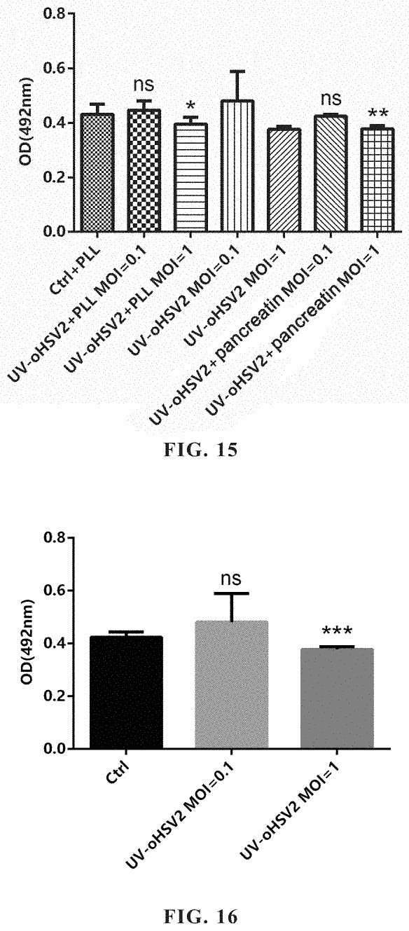

During the experiment, UV-oHSV2 stimulated mouse PBMC was co-incubated with 4T1, the multiplicity of infection was 10:1, and the results are as shown in FIG. 15 and FIG. 16. From FIG. 15, it can be found that, when MOI=1, there is a significant difference between the UV-oHSV2+PLL group and the control group, suggesting that PBMC has a killing effect on 4T1 tumor cells, which is not due to the adherence effect of PBMC to 4T1 cell infection. From FIG. 16, it can be seen that, when MOI=1, UV-oHSV2 stimulated mouse PBMC has a strong killing effect on 4T1 breast cancer, and when MOI=0.1, the UV-oHSV2 stimulated mouse PBMC has no obvious killing effect to 4T1, suggesting that at the time of high multiplicity of infection MOI=1, the effect of the UV-oHSV2 stimulated mouse PBMC in killing tumor is better.

Experimental Example 9. In-vivo killing of CT26 tumor cells by UV-oHSV2 stimulated mouse PBMC

9.1. Preparation of CT26 Cells

Mouse colon cancer cells CT26 were cultivated according to the conventional method, and the medium was DMEM/F12 containing 10% newborn bovine serum. Before tumor induction, the cells were collected, and centrifuged under 2800 rpm for 3 minutes, finally the cell precipitate was re-suspended with a serum-free DMEM/F12 medium, and the cell density was 2×10⁶ cell/ml, ready for use.

9.2. Setting of Dosing Regimen

In this experiment, the animals were treated for three times with UV-oHSV2 stimulated mouse PBMC, and treated once every other two days, totally treated for three times, the detailed specific scheme is seen in the following Table 7.

TABLE 7
Dosing regimen of CT26 tumor model animal experiment

| | Animal | | | | |
|---|---|---|---|---|---|
| | number in | | Sample | Volume | Injection |
| Groups | every group | Sample | concentration | (L) | time (day) |
| Experiment | 3 | UV-oHSV2 | Cell density at 48 | 100 | 0, 3, 6 |
| group | | stimulated | h when stimulating | | |
| | | PBMC | PBMC by UV-oHSV2 | | |
| Blank | 3 | Physiological | — | 100 | 0, 3, 6 |
| control | | saline | | | |
| group | | | | | |

9.3. First Treatment of CT26 with UV-oHSV2 Stimulated Mouse PBMC

1) Preparation of blood sample: blood was collected from the eyeball of the mouse. first treatment: totally 5 ml of blood were collected from 7 mice. The preparation procedure of mouse PBMC was similar to Test Example 2, to obtain a mouse PBMC cell suspension.

2) 1 ml of the obtained mouse PBMC cell suspension was separately taken and added into four wells of a 6-well plate, 40 μL of diabody (1:100) was added in every well, the final volume of every well was 4 ml, the experiment group was infected when MOI=1, the cell density was 2.0×10⁶ cell/ml, and a physiological saline was added in the blank control group. After the planking, the 6-well plate was put in the CO₂ incubator to be cultivated for 48 hours.

3) After 48 hours, the culture media in the holes were mixed, the bottom of the 6-well plate was gently blown and beaten, a small portion of adherent cell was blown and beaten down, the cells were counted, and in every group, 1 ml of the cells was taken ready for use in the animal experiment.

4) the tumor was induced and treated: 6 BALB/c normal mice were divided into 2 groups, 3 mice/group. 100 μl of liquid containing 2×10⁵ CT26 cells was subcutaneously injected at left latus of each of the 6 mice, as the tumor control in every group; in addition, 200 μl of UV-oHSV2 stimulated PBMC+CT26 cell mixed liquor (the mixing ratio was 1:1) was subcutaneously injected at right latus of each of the mice in the experiment group, and 200 μL of physiological saline+CT26 cell mixed liquor (the mixing ratio was 1:1) was injected subcutaneously at right latus of the mice in the blank control group.

9.4. Second Treatment of CT26 with UV-oHSV2 Stimulated Mouse PBMC

1) Preparation of blood sample: blood was collected from the eyeball of the mouse. Second treatment: totally 6 ml of blood were collected from 11 mice. The preparation procedure of mouse PBMC was similar to the Test Example 2, to obtain a mouse PBMC cell suspension.

2) 1 ml of the obtained mouse PBMC cell suspension was separately taken and added into four wells of the 6-well plate, and 40 μL of diabody (1:100) was added into every well, the final volume of every well was 4 ml, the experiment group was infected at MOI=1, and a physiological saline was added in the blank group. After the planking, the 6-well plate was put in the CO₂ incubator to be cultivated for 48 hours.

3) After 48 hours, the culture media in the holes were mixed, the bottom of the 6-well plate was gently blown and beaten, a small portion of adherent cell was blown and beaten down, the cells were counted, and in every group, 1 ml of the cells was taken ready for use in the animal experiment.

4) Second treatment of CT26 tumor: after the tumor in the mice was weighed, in the experiment group, 100 μL of UV-oHSV2 stimulated PBMC was in-situ injected intratumorally only at the right side of each of three mice with bilaterally induced tumor sites, and in the blank control group, 100 μL of physiological saline was injected intratumorally at the right side of each of three mice.

9.5. Third Treatment of CT26 with UV-oHSV2 Stimulated Mouse PBMC

The experiment procedures were in accordance with 9.4.

9.6. Observation

After finish of three times of treatment, the mice were observed and the tumor was weighed. The mice were observed twice every week until the end of the experiment. When the tumor disappeared, CT26 cells with higher cell density were in-situ injected, and the growing status of the tumor was observed.

9.7. Discussion on the Results

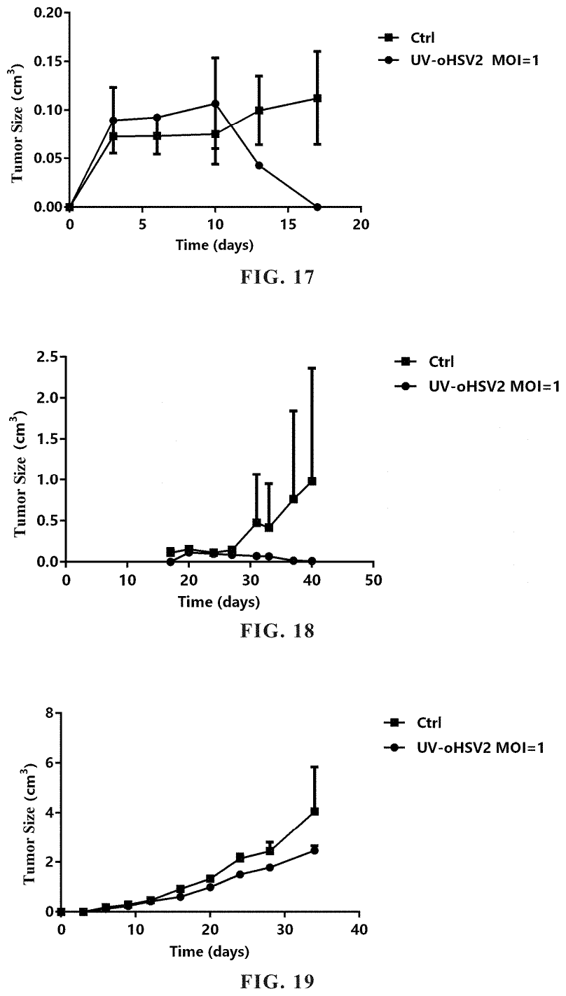

After three times of treatment on day 0, 3, 6 in the experiment, the mice were observed for 40 days, at 17^{th} day of the observation, tumor in 3 mice all disappeared at the treatment site, whereas the mouse tumor size in the blank group always presented a trend of increase, as shown in FIG. 17.

On day 17, after the tumor disappeared, CT26 with a higher cell density (4×10⁶ cells/ml) was re-implanted, tumor implantation was conducted at the original tumor site, tumor was implanted at 100 μl/side for every mouse, the mice were continued to be observed, and observed twice every week. As shown in FIG. 18, with passage of time, it can be found that, when observed until day 40, the tumor in the blank control group presented a trend of continuous increase, whereas in the experiment group UV-oHSV2 (MOI=1) stimulated PBMC treatment group, at the tumor site of the second tumor implantation, a part of the tumor grew out, and a part of the tumor didn't grow out, after growth, a part of the tumor also disappeared with passage of time, suggesting that the UV-oHSV2 (MOI=1) stimulated PBMC treatment group might have a persistent immunological effect, and what immunocytes plays a role is under study.

### Experimental Example 10. In-Vivo Killing of 4T1 Tumor Cells by UV-oHSV2 Stimulated Mouse PBMC

This experimental example investigated the tumor treatment effect of UV-oHSV2 stimulated PBMC in-vivo against the mouse breast cancer 4T1. 4T1 originated from a BALB/c mouse breast cancer cell line has a 6-guanine resistance.

10.1. Preparation of 4T1 Cells

The mouse breast cancer cell (4T1) was cultivated according to the conventional method, and the medium was DMEM/F12 containing 10% fetal bovine serum. Before the tumor induction, the cells were collected, and centrifuged under 2800 rpm for 3 minutes, finally the cell precipitate was re-suspended with a serum-free DMEM/F12 medium, and the cell density was 4×10⁶ cell/ml, ready for use.

10.2. The Dosing Regimen is Seen in Table 8.

TABLE 8
Dosing regimen for 4T1 tumor model animal experiment

| | Animal | | | | |
|---|---|---|---|---|---|
| | number in | | Sample | Volume | Injection |
| Groups | every group | Sample | concentration | (μL) | time (day) |
| Experiment | 5 | UV-oHSV2 | Cell density at 48 | 100 | 0, 3, 6 |
| group | | stimulated | hours when stimulating | | |
| | | PBMC | PBMC by UV-oHSV2 | | |
| Blank | 5 | Physiological | — | 100 | 0, 3, 6 |
| control | | saline | | | |
| group | | | | | |

10.3. 4T1 was treated with UV-oHSV2 stimulated mouse PBMC for three times, and the experiment procedures were similar to those in the Experimental Example 9.3-9.5.

10.4. Observation

After finish of three times of treatment, the mice were observed and the tumors were weighed. The mice were observed twice every week until the experiment finished. When the tumor disappeared, 4T1 cells with higher cell density were in-situ injected, and the growing status of the tumors was observed.

10.5. Discussion on the Results

After three times of treatment on day 0, day 3, and day 6, an observation was conducted for 34 days, the tumor size at the treated side of the mouse is as shown in FIG. 19, it can be seen that the mouse tumor sizes in the control group and the UV-oHSV2 (MOI=1) stimulated PBMC treatment group all present a trend of increase, but the mouse tumor size in the UV-oHSV2 (MOI=1) stimulated PBMC treatment group increased more slowly (only a half of the size in the blank control group), there is no significant difference compared with the blank control group, but based on the observation from the tumor growth trend, the UV-oHSV2 (MOI=1) stimulation group has a certain inhibiting effect against growth of the tumor.

### Experimental Example 11. Study on Human PBMC Proliferation Experiment

The bloods of human were from the volunteers SXT, LBL, KZH, and GQX in this laboratory, and four groups were set, and were respectively UV-oHSV2 (MOI=0.1), UV-oHSV2 (MOI=1), phytohaemagglutinin (PHA), and Ctrl.

Phytohaemagglutinin (PHA) is a mitogen, and is mainly used to inactivate the immunocytes-lymphocyte.

11.1. Preparation of Blood Sample

The experiment procedures were similar to those in Test Example 4.1, the difference is in that, the set groups are seen in the following Table 9.

TABLE 9
Scheme of human PBMC planking

| | Groups (unit: μL) | | | |
|---|---|---|---|---|
| | | | UV-oHSV2 | UV-oHSV2 |
| | Ctrl | PHA | MOI = 1 | MOI = 0.1 |
| Human PBMC | 500 | 500 | 500 | 500 |
| PHA | 0 | 5 | 0 | 0 |
| Inactivated virus | 0 | 0 | 200 | 20 |
| Inactivated plasma | 200 | 200 | 200 | 200 |
| 100 × diabody | 20 | 20 | 20 | 20 |
| DMEM/F12 | 1280 | 1275 | 1080 | 1260 |

11.2. Experimental Results

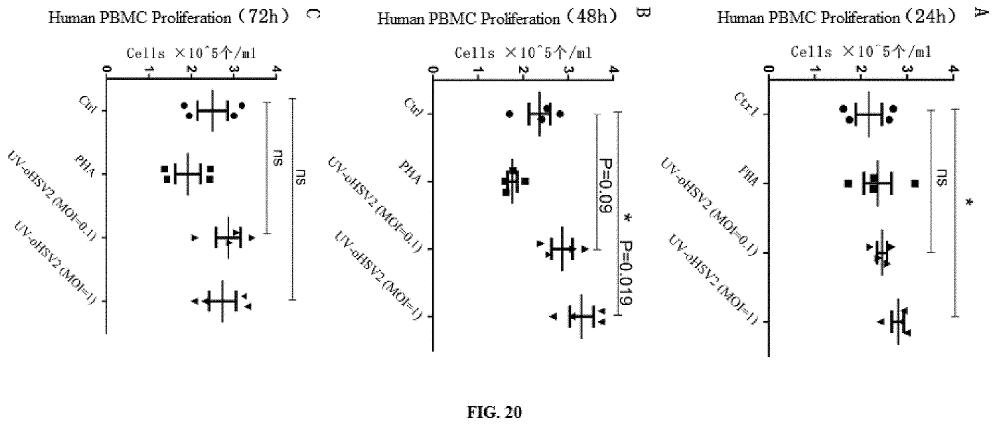

At 24 h, 48 h and 72 h, the stimulated PBMC in each group was counted by a cell counter, the statistical data are as shown in the following FIG. 20, every point represents one independent experiment, and statistical analysis is conducted on the results obtained by the experiment by using GraphPad Prism 6.0 software. The comparison between the groups adopts t-test, the results were analyzed on the basis of P<0.05 having a statistical difference.

11.3. Discussion on Results

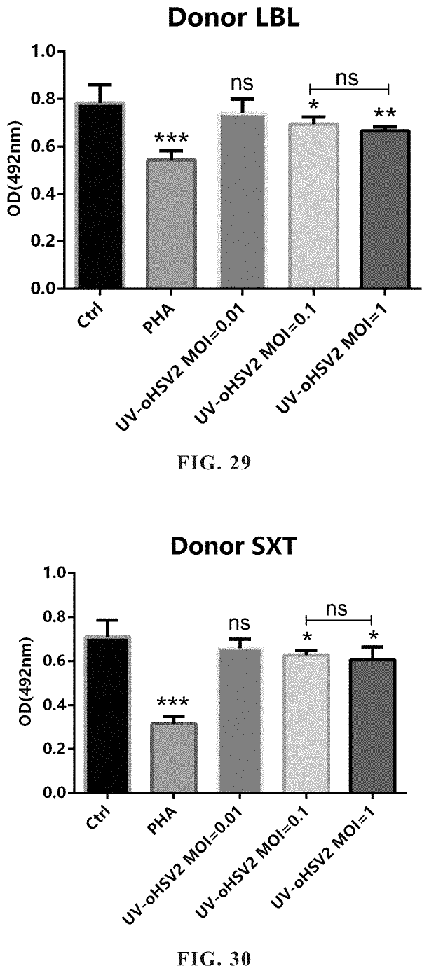

This proliferation experiment adopted the fresh bloods from 4 healthy volunteers, in FIG. 20, every point represents one independent experiment, it can be seen that, when MOI=0.1, the UV-oHSV2 stimulated PBMC at 24 hours and 72 hours has no obvious proliferation, at 48 hours, there is no significant difference compared with the control group, but there is a trend of proliferation, and there are also a certain of individual difference among volunteers. When MOI=1, UV-oHSV2 stimulated PBMC at 24 h and 48 h both have obvious proliferation effects. But the positive stimulator PHA at 24 h, 48 h and 72 h all have no obvious proliferation (but the positive stimulator has a good killing effect when killing the tumor cells, and the results were seen in FIG. 22 to FIG. 30.

### Experimental Example 12. In-Vitro Killing of LoVo Tumor Cells by UV-oHSV2 Simulated Human PBMC

This experimental example investigated whether the stimulated human PBMC can be activated after 48 hours of proliferation, and whether in-vitro killing of tumor cells by the activated PBMC was related to MOI of oHSV2 or the degree of proliferation.

This experiment adopted human colon cancer (LoVo) cells, and explored whether the PBMC after the proliferation activation had a killing effect on LoVo cells by MTT colorimetry. The blood samples from four volunteers LBL, GXQ, ST, and SXT were used as the experiment subjects.

12.1. Planking of human PBMC, the experiment procedure was similar to the Test Example 4.1, and the difference is in that, the set groups were similar to those in Table 9.

After the planking, a 6-well plate was put in the CO₂ incubator to be cultivated for 48 hours, after 48 hours, the culture media in the holes were mixed, the bottom of the 6-well plate was gently blown and beaten, a small portion of the adherent cells were blown and beaten down, the cells were counted with the cell counter, the cell densities in every group at 48 h were different, in order to unify the multiplicity of infection, the PBMC cell densities in every group were diluted to the same density, ready for use. That is, by ignoring the degree of PBMC proliferation in each group, the degree of PBMC activation in each group was analyzed at the same cell density.

12.2. Preparation of LoVo Cells

LoVo cells were cultivated by the conventional technique, the medium was removed after the culture, the cells were re-suspended and counted, and the cells were diluted to a cell density of 2×10⁵ cell/ml with DMEM/F12 containing 10% fetal bovine serum, ready for use.

12.3 Coating of the Polylysine (PLL) Plate: Conventional Technique.

12.4 LoVo Cell In-Vitro Killing Planking

1) The experiment was divided into seven groups, and every group had 5 holes, totally 150 μl/well of mixed liquor.

2) Experiment group one: UV-oHSV2 stimulated PBMC when MOI=0.1, 1 50 μl+LoVo 50 μl+FBS containing 10% DMEM/F12 50 μl (a well containing PLL, PLL is used as an adherence promoter).

3) Experiment group two: 50 μl of UV-oHSV2 stimulated PBMC when MOI=0.1, 1+50 μl of LoVo+50 μl of DMEM/F12 containing 10% FBS.

4) Experiment group three: 50 μl of UV-oHSV2 stimulated PBMC when MOI=0.1, 1+50 μl of LoVo+50 μl of DMEM/F12 containing 10% FBS+50 μl of 5-fold diluted pancreatin (pancreatin is used as an adherence inhibitor).

5) Experiment group four: 50 μl of PHA (a positive stimulator) stimulated PBMC+50 μl of LoVo+50 μl of DMEM/F12 containing 10% FBS.

6) Experiment group five: blank group (containing PBMC) 50 μl+50 μl of LoVo+50 μl of DMEM/F12 containing 10% FBS.

7) Experiment group six: 50 μl of LoVo+100 μl of DMEM/F12 containing 10% FBS (a well containing PLL).

8) Blank group: 50 μl of LoVo+100 μl of DMEM/F12 containing 10% FBS.

9) After the planking, the 96-well plate was put in the CO₂ incubator to be cultivated for 48 to 72 hours.

12.5. MTT Detection

12.6. The Experimental Results are as Show in the Following Table.

TABLE 10
Table of results of in-vitro killing LoVo
in different groups for volunteer LBL

| | | | | | | Mean |
|---|---|---|---|---|---|---|
| Groups | OD value | | | | | value |
| UV-HSV2 (PBMC) + | 0.197 | 0.250 | 0.217 | 0.230 | 0.267 | 0.232 |
| PLL MOI = 0.1 | | | | | | |
| UV-HSV2 (PBMC) + | 0.200 | 0.189 | 0.217 | 0.188 | 0.193 | 0.197 |
| PLL MOI = 1 | | | | | | |
| UV-HSV2 (PBMC) | 0.210 | 0.228 | 0.214 | 0.262 | 0.260 | 0.235 |
| MOI = 0.1 | | | | | | |
| UV-HSV2 (PBMC) | 0.187 | 0.219 | 0.212 | 0.235 | 0.218 | 0.214 |
| MOI = 1 | | | | | | |
| UV-HSV2 (PBMC) + | 0.284 | 0.312 | 0.265 | 0.268 | 0.260 | 0.278 |
| pancreatin MOI = 0.1 | | | | | | |
| UV-HSV2 (PBMC) + | 0.190 | 0.186 | 0.190 | 0.193 | 0.196 | 0.191 |
| pancreatin MOI = 1 | | | | | | |
| PHA (PBMC) | 0.221 | 0.239 | 0.225 | 0.213 | 0.190 | 0.218 |
| Ctrl (PBMC) | 0.331 | 0.347 | 0.374 | 0.361 | 0.360 | 0.355 |
| Ctrl + PLL | 0.414 | 0.407 | 0.404 | 0.380 | 0.365 | 0.394 |
| Ctrl | 0.343 | 0.339 | 0.341 | 0.341 | 0.353 | 0.343 |

TABLE 11
Table of results of in-vitro killing LoVo
in different groups for volunteer GQX

| | | | | | | Mean |
|---|---|---|---|---|---|---|
| Groups | OD value | | | | | value |
| UV-HSV2 (PBMC) + | 0.221 | 0.223 | 0.251 | 0.275 | 0.239 | 0.242 |
| PLL MOI = 0.1 | | | | | | |
| UV-HSV2 (PBMC) + | 0.199 | 0.222 | 0.262 | 0.217 | 0.248 | 0.230 |
| PLL MOI = 1 | | | | | | |
| UV-HSV2 (PBMC) | 0.247 | 0.274 | 0.269 | 0.238 | 0.278 | 0.261 |
| MOI = 0.1 | | | | | | |
| UV-HSV2 (PBMC) | 0.221 | 0.243 | 0.257 | 0.264 | 0.217 | 0.240 |
| MOI = 1 | | | | | | |
| UV-HSV2 (PBMC) + | 0.233 | 0.269 | 0.263 | 0.250 | 0.216 | 0.246 |
| pancreatin MOI = 0.1 | | | | | | |
| UV-HSV2 (PBMC) + | 0.260 | 0.258 | 0.239 | 0.248 | 0.248 | 0.251 |
| pancreatin MOI = 1 | | | | | | |
| PHA (PBMC) | 0.201 | 0.221 | 0.248 | 0.198 | 0.213 | 0.216 |
| Ctrl (PBMC) | 0.398 | 0.453 | 0.382 | 0.412 | 0.443 | 0.418 |
| Ctrl + PLL | 0.419 | 0.466 | 0.414 | 0.429 | 0.428 | 0.431 |
| Ctrl | 0.366 | 0.401 | 0.359 | 0.392 | 0.400 | 0.384 |

TABLE 12
Table of results of in-vitro killing LoVo
in different groups for volunteer ST

| | | | | | | Mean |
|---|---|---|---|---|---|---|
| Groups | OD value | | | | | value |
| UV-HSV2 (PBMC) | 0.301 | 0.326 | 0.307 | 0.297 | 0.294 | 0.305 |
| MOI = 0.1 | | | | | | |
| UV-HSV2 (PBMC) | 0.210 | 0.251 | 0.226 | 0.204 | 0.233 | 0.225 |
| MOI = 1 | | | | | | |
| PHA (PBMC) | 0.215 | 0.248 | 0.212 | 0.220 | 0.207 | 0.220 |
| Ctrl (PBMC) | 0.407 | 0.437 | 0.458 | 0.487 | 0.492 | 0.456 |
| Ctrl | 0.388 | 0.378 | 0.409 | 0.388 | 0.371 | 0.387 |

TABLE 13
Table of results of in-vitro killing LoVo
in different groups for volunteer SXT

| | | | | | | Mean |
|---|---|---|---|---|---|---|
| Groups | OD value | | | | | value |
| UV-HSV2 (PBMC) | 0.745 | 0.341 | 0.271 | 0.327 | 0.254 | 0.298 |
| MOI = 0.1 | | | | | | |
| UV-HSV2 (PBMC) | 0.246 | 0.235 | 0.244 | 0.293 | 0.233 | 0.250 |
| MOI = 1 | | | | | | |
| PHA (PBMC) | 0.151 | 0.188 | 0.200 | 0.200 | 0.198 | 0.187 |
| Ctrl (PBMC) | 0.436 | 0.441 | 0.519 | 0.438 | 0.404 | 0.448 |
| Ctrl | 0.395 | 0.457 | 0.390 | 0.420 | 0.378 | 0.408 |

12.7 Discussion on Results

A PLL adherence promoter group and a pancreatin adherence inhibitor group were added in the experiment, to explore when UV-oHSV2 stimulated PBMC was co-incubated with the tumor cells in the experiment group, the killing effect is not because UV-oHSV2 stimulated PBMC interrupts the adherence of the tumor cell, but because the stimulated PBMC has the ability of killing tumor cells after activation, and the experimental results also just demonstrate this viewpoint.

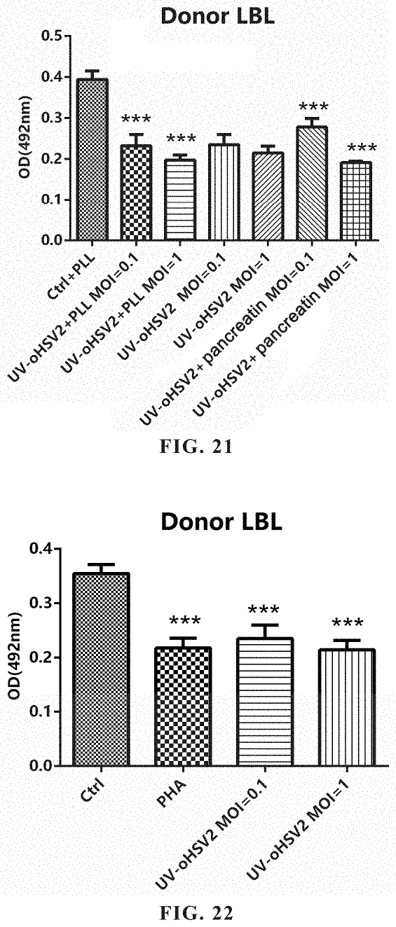

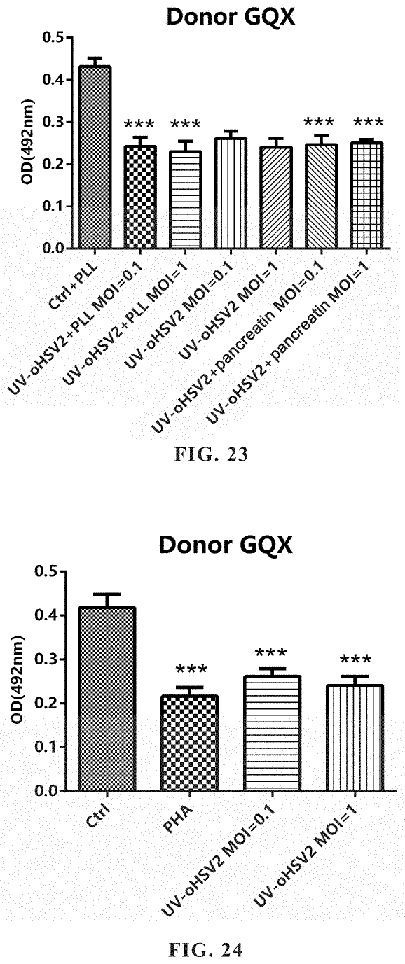

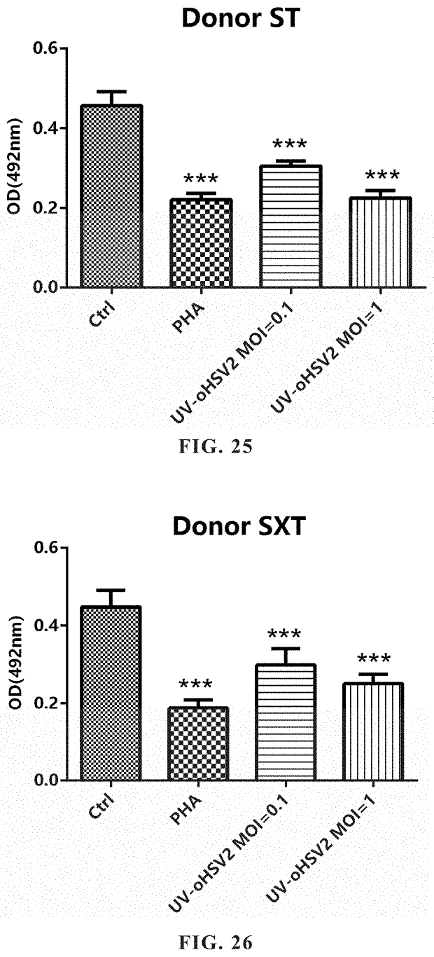

As shown in FIG. 21 and FIG. 23, from the experimental results of two different volunteers, it can be seen that, even in the presence of the adherence promoter PLL, the UV-oHSV2 stimulation group still has a strong killing effect when MOI=0.1 and MOI=1. From FIG. 22, FIG. 24, FIG. 25 and FIG. 26, it can be seen that, when MOI=0.1 and MOI=1, UV-oHSV2 stimulated PBMC of four different volunteers have strong killing results, and at high multiplicity of infection, the killing effect is stronger.

### Experimental Example 13. In-Vitro Killing of BGC823 Tumor Cells by UV-oHSV2 Stimulated Human PBMC

13.1. The experiment step is similar to Experimental Example 12, and blood samples from four volunteers GQX, LBL, KZH, and SXT were selected as the experiment subjects.

13.2. The experimental results are as shown in the following table.

TABLE 14
Table of results of in-vitro killing BGC823
in different groups for volunteer GQX

| | | | | | | Mean |
|---|---|---|---|---|---|---|
| Groups | OD value | | | | | value |
| UV-HSV2 (PBMC) | 0.870 | 0.905 | 0.949 | 0.865 | 0.994 | 0.917 |
| MOI = 0.01 | | | | | | |
| UV-HSV2 (PBMC) | 0.688 | 0.708 | 0.683 | 0.728 | 0.788 | 0.719 |
| MOI = 0.1 | | | | | | |
| UV-HSV2 (PBMC) | 0.586 | 0.561 | 0.537 | 0.596 | 0.554 | 0.567 |
| MOI = 1 | | | | | | |
| PHA (PBMC) | 0.935 | 0.950 | 0.925 | 0.931 | 0.923 | 0.933 |
| Ctrl | 0.932 | 0.938 | 0.896 | 0.936 | 1.011 | 0.943 |

TABLE 15
Table of results of in-vitro killing BGC823
in different groups for volunteer KZH

| | | | | | | Mean |
|---|---|---|---|---|---|---|
| Groups | OD value | | | | | value |
| UV-HSV2 (PBMC) | 0.853 | 0.928 | 0.939 | 0.952 | 0.981 | 0.9306 |
| MOI = 0.01 | | | | | | |
| UV-HSV2 (PBMC) | 0.828 | 0.93 | 0.957 | 0.92 | 0.916 | 0.9102 |
| MOI = 0.1 | | | | | | |
| UV-HSV2 (PBMC) | 0.934 | 0.847 | 0.841 | 0.923 | 0.932 | 0.8954 |
| MOI = 1 | | | | | | |
| PHA (PBMC) | 0.693 | 0.852 | 0.882 | 0.842 | 0.878 | 0.8294 |
| Ctrl | 0.933 | 1.001 | 0.967 | 1.005 | 1.051 | 0.9914 |

TABLE 16
Table of results of in-vitro killing BGC823
in different groups for volunteer LBL

| | | | | | | Mean |
|---|---|---|---|---|---|---|
| Groups | OD value | | | | | value |
| UV-HSV2 (PBMC) | 0.755 | 0.788 | 0.739 | 0.777 | 0.639 | 0.740 |
| MOI = 0.01 | | | | | | |
| UV-HSV2 (PBMC) | 0.706 | 0.740 | 0.661 | 0.685 | 0.678 | 0.694 |
| MOI = 0.1 | | | | | | |
| UV-HSV2 (PBMC) | 0.639 | 0.676 | 0.658 | 0.678 | 0.678 | 0.666 |
| MOI = 1 | | | | | | |
| PHA (PBMC) | 0.533 | 0.494 | 0.600 | 0.557 | 0.533 | 0.543 |
| Ctrl | 0.691 | 0.746 | 0.753 | 0.836 | 0.884 | 0.782 |

TABLE 17
Table of results of in-vitro killing BGC823
in different groups for volunteer SXT

| | | | | | | Mean |
|---|---|---|---|---|---|---|
| Groups | OD value | | | | | value |
| UV-HSV2 (PBMC) | 0.611 | 0.640 | 0.674 | 0.718 | 0.649 | 0.658 |
| MOI = 0.01 | | | | | | |
| UV-HSV2 (PBMC) | 0.603 | 0.616 | 0.636 | 0.627 | 0.655 | 0.627 |
| MOI = 0.1 | | | | | | |
| UV-HSV2 (PBMC) | 0.553 | 0.627 | 0.606 | 0.691 | 0.555 | 0.606 |
| MOI = 1 | | | | | | |
| PHA (PBMC) | 0.265 | 0.348 | 0.340 | 0.322 | 0.304 | 0.316 |
| Ctrl | 0.704 | 0.721 | 0.828 | 0.669 | 0.627 | 0.710 |

13.3. Discussion on Results

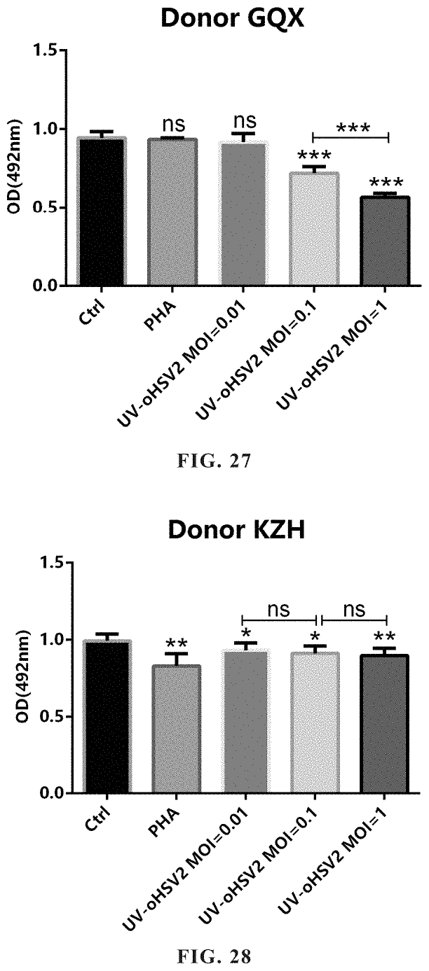

As shown in FIG. 27 to FIG. 30, from the experimental results of four different volunteers, it can be seen that, UV-oHSV2 stimulation group has a strong killing effect against BGC823 tumor cell when MOI=0.1 and MOI=1. And at high mutiplicity of infection, the killing effect is higher. And in volunteer GQX, when MOI=1 the killing effect of UV-oHSV2 of the stimulated PBMC is better than that of the positive stimulator PHA.

### Experiment Example 14. In-Vivo Killing of LoVo Tumor Cells by UV-oHSV2 Stimulated Human PBMC

Experimental Example 12 investigated the in-vitro killing of LoVo tumor cells by human PBMC, and this experimental example investigated the in-vivo killing effect.

14.1. Preparation of LoVo Cells

The human colon cancer LoVo cells were cultivated according to the conventional method, and the medium was DMEM/F12 containing 10% fetal bovine serum. Before the tumor induction, the cells were collected, and centrifuged under 2800 rpm for 3 minutes, finally the cell precipitate was re-suspended with a serum-free DMEM/F12 medium, and the cell density was 1×10⁷ cell/ml, ready for use.

14.2. Setting of Dosing Regimen

This experiment adopted UV-oHSV2 stimulated Human PBMC treatment for three times, treated once every three days, totally treated for three times, the detailed specific scheme is seen in the following table.

TABLE 18
Dosing regimen of LoVo tumor model animal experiment

| | Animal | | | | |
|---|---|---|---|---|---|
| | number in | | Sample | Volume | Injection |
| Groups | every group | Sample | concentration | (L) | time (day) |
| UV-HSV2 | 3 | UV-oHSV2 | Cell density at 48 | 100 | 0, 3, 6 |
| (MOI = 0.01) | | (MOI = 0.01) | h when stimulating | | |
| PBMC | | stimulated | PBMC by UV-oHSV2 | | |
| | | PBMC | (MOI = 0.01) | | |
| UV-HSV2 | 3 | UV-oHSV2 | Cell density at 48 | 100 | 0, 3, 6 |
| (MOI = 0.1) | | (MOI = 0.1) | h when stimulating | | |
| PBMC | | stimulated | PBMC by UV-oHSV2 | | |
| | | PBMC | (MOI = 0.1) | | |
| UV-HSV2 | 3 | UV-oHSV2 | Cell density at 48 | 100 | 0, 3, 6 |
| (MOI = 1) | | (MOI = 1) | h when stimulating | | |
| PBMC | | stimulated | PBMC by UV-oHSV2 | | |
| | | PBMC | (MOI = 1) | | |
| PHA | 3 | PHA | Cell density at 48 | 100 | 0, 3, 6 |
| (PBMC) | | stimulated | h when stimulating | | |
| | | PBMC | PBMC by PHA | | |
| Ctrl | 3 | Blank | Cell density at 48 | 100 | 0, 3, 6 |
| (PBMC) | | stimulated | h when stimulating | | |
| | | PBMC | PBMC by blank | | |
| Blank | 3 | Physiological | — | 100 | 0, 3, 6 |
| control | | saline | | | |
| group | | | | | |

14.3 Treatment of LoVo by U V-oHSV2 Stimulated Human PBMC for Three Times

The experiment procedures were in accordance with Experimental Example 9.3-9.5, the difference is in that, the experiment mouse was BALB/c-nu nude mouse, the experiment groups were divided into three classes (they are respectively MOI=0.01/0.1/1), LoVo tumor implantation density: every mouse was injected with 100 μL of cell liquid containing 1×10⁶ LoVo.

14.4 Observation

After completion of three times of treatment, the mouse was observed, and the tumor was weighed. The mouse was observed twice every week until completion of the experiment. When the tumor disappeared, the LoVo cells with higher cell density were in-situ injected, and the growing status the tumor was observed.

14.5. Experimental Results

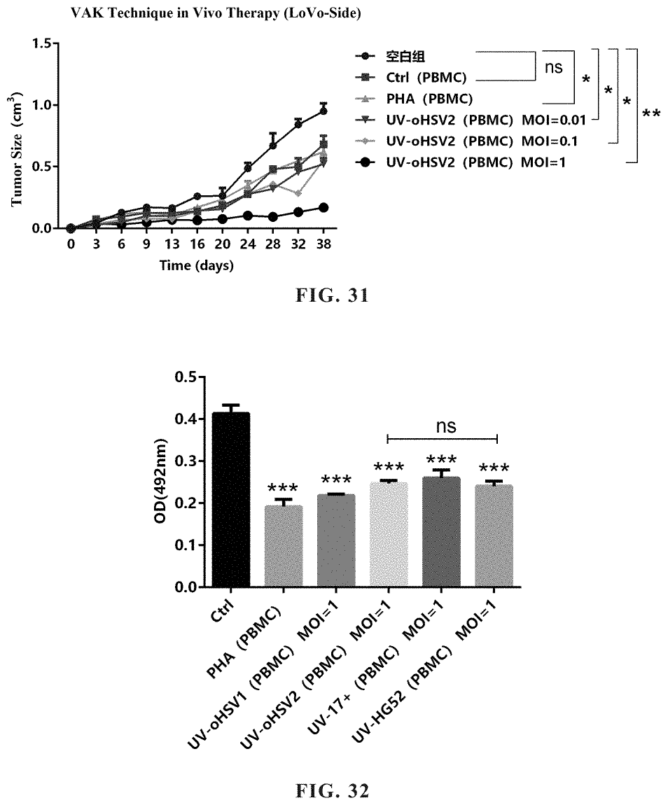

In the experiment, after three times of treatment on day 0, 3, 6, the mouse was observed for 38 days, as shown in FIG. 31, it can be found that, with passage of time, when the mouse was observed until day 38, the tumor in the blank control group presents a trend of continuous increase, whereas in the experiment group UV-oHSV2 (MOI=1) stimulated PBMC treatment group, an obvious inhibiting effect is achieved on tumor with passage of time (P=0.0035048). PHA, UV-oHSV2 (MOI=0.01/0.1) stimulated PBMC treatment groups likewise had an effect of inhibiting increase of the LoVo tumor cells (P values were respectively: P=0.026, P=0.0186, P=0.0196), and there is no statistical difference between the Ctrl stimulated PBMC treatment group and the blank control group (P=0.069>0.05).

The above-described experimental example proved that UV-oHSV2 stimulated and proliferated human and mouse PBMC both can effectively kill tumor cells in-vivo and in-vitro.

In the in-vitro experiment, UV-oHSV2 stimulated mouse PBMC is most obvious at 48 hours, when the sample at 48 h was adopted and MOI=1, mouse colon cancer CT26 and mouse breast cancer 4T1 can be killed well. In the human PBMC stimulation proliferation experiment, the PBMCs from four different volunteers were respectively adopted, when the MOI of the UV-oHSV2 was equal to 1, proliferation was likewise most obvious at 48 h, when the sample at 48 h was adopted and MOI=1, PBMCs from four different volunteers have good killing results on human colon cancer LoVo tumor cells and human gastric cancer BGC823 tumor cells when MOI=0.1 and MOI=1.

In the in-vivo experiment, it can be seen that, UV-oHSV2 stimulated PBMC, at 48 h, by treating the CT26 tumor for three times in succession, can make the tumor completely disappear on day 17, the CT26 tumor cell of higher dose was again in-situ injected, and the tumor finally still disappeared, suggesting that UV-oHSV2 stimulated mouse PBMC has a persistent immunotherapeutic effect. When the 4T1 tumor cell is treated in-vivo, UV-oHSV2 stimulated mouse PBMC can inhibit the tumor increase. The above suggests that in the in-vivo experiment, UV-oHSV2 stimulated mouse PBMC can inhibit growth of the tumor cells, even make the tumor cell disappear.

### Experimental Example 15

This experiment investigated in-vitro killing of tumor cells by ultraviolet inactivated oHSV2 (1E7 CCID₅₀/ml), oHSV1 (1E7 CCID₅₀/ml), wild-type HSV1 (17⁺, 1E8 CCID₅₀/ml), wild-type HSV2 (HG52, 1E7 CCID₅₀/ml) (MOI=1) stimulated human PBMC after 48 h of activation. This experiment adopted human colon cancer (LoVo) cells, and explored whether the PBMC after proliferation and activation may kill LoVo cells or not by MTT colorimetry.

15.1. The planking of the human PBMC was similar to Test Example 4.1 (Donor: FZY), the difference is in that, the oHSV2 in the step was replaced with the above described 4 classes, and the set groups were seen in the following table.

TABLE 19
Planking scheme of Human PBMC

| | Groups (unit: μL) | | | | | |
|---|---|---|---|---|---|---|
| | | | UV- | UV- | UV-17⁺ | UV- |
| | | | oHSV1 | oHSV2 | MOI = | HG52 |
| | Ctrl | PHA | MOI = 1 | MOI = 1 | 1 | MOI = 1 |
| Human | 1000 | 1000 | 1000 | 1000 | 1000 | 1000 |
| PBMC | | | | | | |
| PHA | 0 | 5 | 0 | 0 | 0 | 0 |
| Inactivated | 0 | 0 | 200 | 200 | 20 | 200 |
| virus | | | | | | |
| Inactivated | 200 | 200 | 200 | 200 | 200 | 200 |
| plasma | | | | | | |
| 100 × | 20 | 20 | 20 | 20 | 20 | 20 |
| diabody | | | | | | |
| DMEM/ | 780 | 775 | 580 | 580 | 760 | 580 |
| F12 | | | | | | |

After planking, the 6-well plate was put in the CO₂ incubator to be cultivated for 48 hours. After 48 hours, the culture media in the holes were mixed, the bottom of the 6-well plate was gently blown and beaten, a small portion of adherent cell was blown and beaten down, and the cells were counted by a cell counter, the cell densities in every group at 48 hours were different, in order to unify the multiplicity of infection, the PBMC cell density in every group was diluted to the same density, ready for use. Namely, ignoring degree of PBMC proliferation in every group, the degree of PBMC activation was analyzed in each group at the same cell density.

15.2. Preparation of LoVo Cells

LoVo cells were cultivated by the conventional technique, after the culture, the medium was removed, the cells were re-suspended and counted, and the cells were diluted to a cell density of 2.5×10⁵ cell/ml with DMEM/F12 containing 10% fetal bovine serum, ready for use.

15.3 LoVo Cell In-Vitro Killing Planking

1) The experiment was divided into seven groups, and every group had 5 holes, totally 100 μl/well of mixed liquor.

2) Experiment group one: 50 μl of UV-oHSV1 stimulated PBMC when MOI=1+50 μl of LoVo.

3) Experiment group two: 50 μl of UV-oHSV2 stimulated PBMC when MOI=1+50 μl of LoVo.

4) Experiment group three: 50 μl of UV-17+stimulated PBMC when MOI=1+50 μl of LoVo.

5) Experiment group four: 50 μl of UV-HG52 stimulated PBMC when MOI=1+50 μl of LoVo.

6) Experiment group five: 50 μl of PHA (a positive stimulator) stimulated PBMC+50 μl of LoVo.

7) Experiment group six: blank group (containing PBMC) 50 μl+50 μl of LoVo.

8) Experiment group seven: blank group, 50 μl of LoVo+DMEM/F12 containing 10% FBS.

9) After the planking, the 96-well plate was put in the CO₂ incubator to be cultivated for 48 to 72 hours.

15.4 MTT Assay

15.5 The experimental result data are as seen in the following table, and seen in FIG. 32.

TABLE 20
Table of results of in-vitro killing LoVo
in different groups for volunteer FZY

| | | | | | | Mean |
|---|---|---|---|---|---|---|
| Groups | OD value | | | | | value |
| UV-HSV1 (PBMC) | 0.214 | 0.220 | 0.216 | 0.223 | 0.219 | 0.218 |
| MOI = 1 | | | | | | |
| UV-HSV2 (PBMC) | 0.244 | 0.239 | 0.256 | 0.242 | 0.254 | 0.247 |
| MOI = 1 | | | | | | |
| UV-17⁺ (PBMC) | 0.261 | 0.239 | 0.286 | 0.269 | 0.243 | 0.260 |
| MOI = 1 | | | | | | |
| UV-HG52 (PBMC) | 0.232 | 0.233 | 0.261 | 0.243 | 0.235 | 0.241 |
| MOI = 1 | | | | | | |
| PHA (PBMC) | 0.166 | 0.199 | 0.209 | 0.202 | 0.184 | 0.192 |
| Ctrl (PBMC) | 0.403 | 0.448 | 0.405 | 0.401 | 0.411 | 0.414 |
| Ctrl | 0.367 | 0.371 | 0.358 | 0.363 | 0.378 | 0.367 |

The above described experimental results show that, the herpes simplex virus has excellent characteristics of in-vitro activating immunocytes, whether wild-type or recombinant type, type I or type II all have the effect of activating the immunocytes. It is concluded that, such viruses have universal characteristics of activating immunocytes.

### Experimental Example 16

Detection of Activation of CD4⁺T Cells and NK Cells in Human VAK

16.1. Planking of Human VAK

Similar to Test Example 4, the difference is in that, the volunteer was only SXT, and the PBMC was incubated with the inactivated virus for 48 hours.

16.2. Isolation of T Cells and NK Cells

1) NK cells and T cells were isolated by using negative selecting magnetic beads from BD company.

2) By the conventional method, NK cells were respectively labeled with human CD3, CD56, and CD69; and T cells were labeled with CD3, CD4, CD8 and CD25.

3) Activation of NK cells, CD4⁺T cells and CD8+T cells were respectively detected by the flow cytometry.

16.3. Experimental Result and Discussion

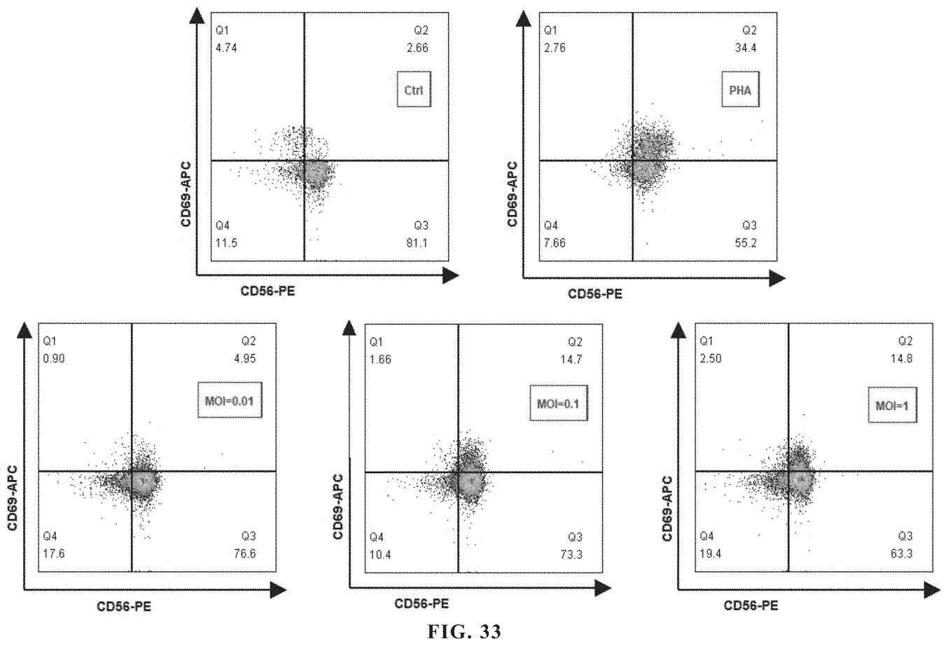

Experimental results are as shown in FIG. 33 to FIG. 35, it can be known from FIG. 33 that when MOI=0.1 and MOI=1, NK cells can be significantly activated, and PHA is the positive control.

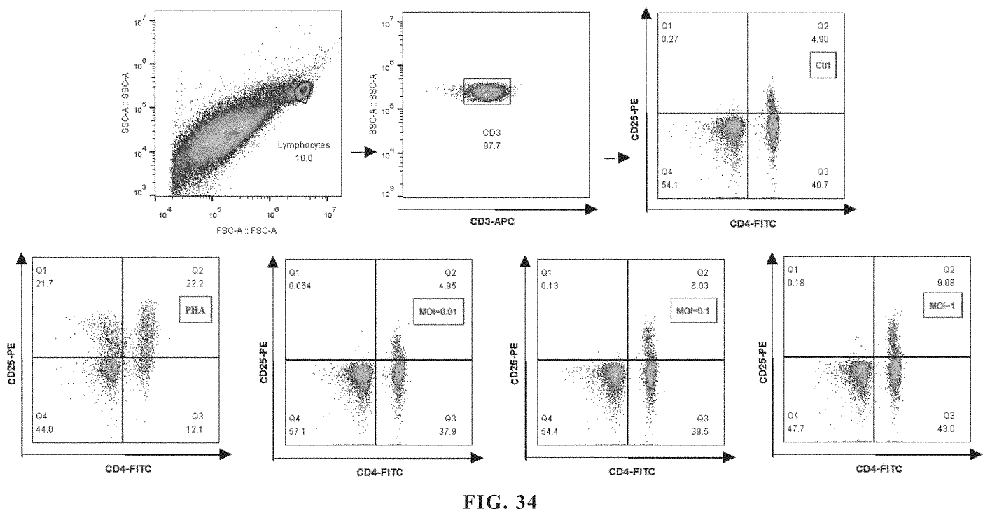

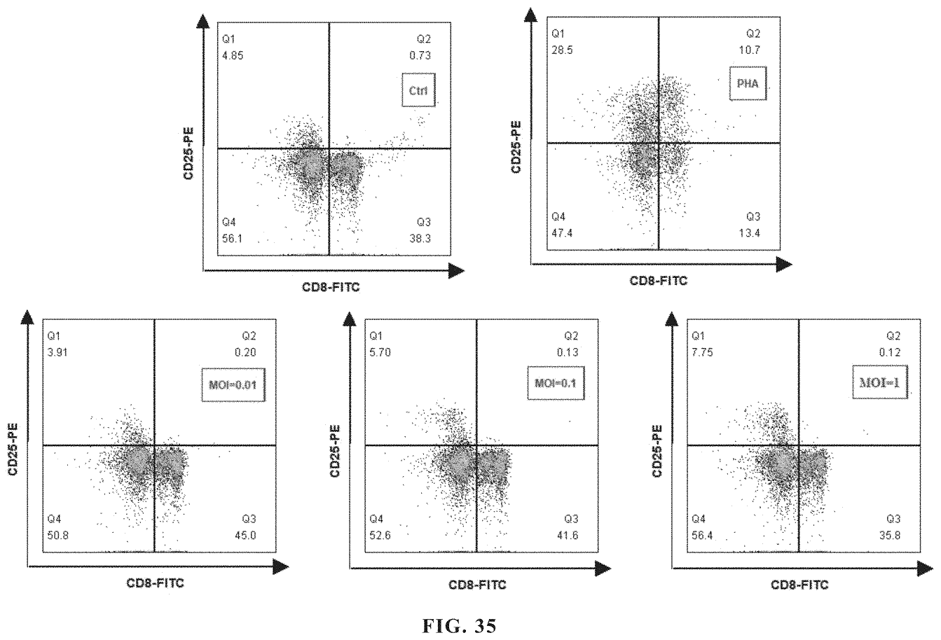

It can be known from FIG. 34 and FIG. 35 that when MOI=1, CD4⁺T cells in VAK can be activated, and CD8⁺T cells can not be activated.

### Experimental Example 17

Flow Detection of Killing LOVO by Activated NK in VAK

2.1 Human VAK planking: similar to Test Example 4, the difference is in that PBMC and inactivated virus were incubated for 48 hours.

2.2. Isolation of NK Cells in VAK: Similar to Experimental Example 16.2.

2.3. Planking of Killing LOVO by Isolated NK

1) The LOVO cells used in the experiment were the LOVO-GFP cell line after gene modification, and green light could be detected in the flow cytometer; the cell planking density was 2×10⁴ cell/well, and a 96-well plate was used.

2) The isolated NK cell was co-incubated with LOVO-GFP cells in a 37° C., 5% CO₂ incubator for 24 hours, and cell apoptosis signals of annexin V and 7-AAD were detected through upflow detection.

2.4 Experimental Results and Discussion

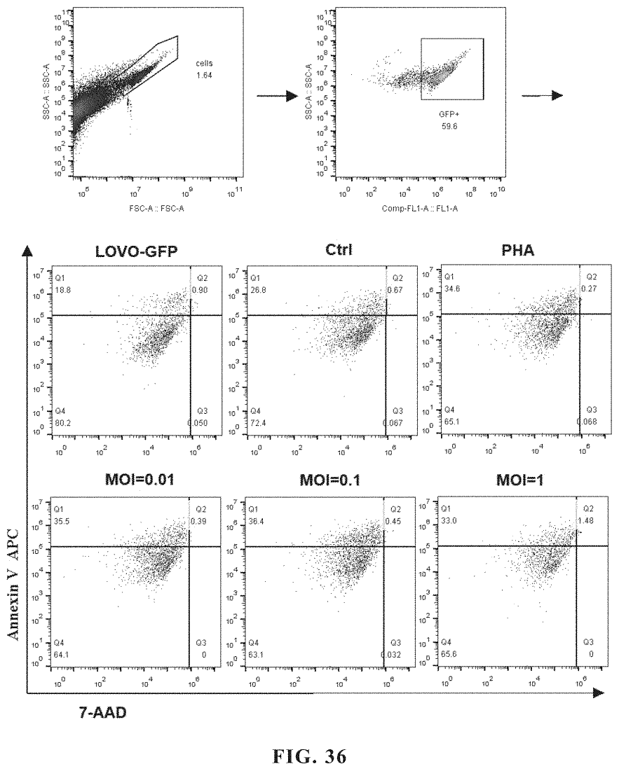

It can be known from the experimental results of 16.3 that the inactivated virus can activate NK cells in VAK, whereas in FIG. 36, the activated NK cells in this experiment likewise has the oncolytic effect against LOVO, and can induce apoptosis of the LOVO cells.

What is claimed is:

1. A method for activating immunocytes in vitro by VAK technique, comprising the following steps:
(1) Isolating immunocytes from peripheral blood or malignant pleural effusion samples of a patient;
(2) Co-incubating inactivated herpes simplex viruses with the immunocytes, to activate the immunocytes; and
(3) Removing the inactivated herpes simplex viruses to obtain the activated immunocytes.

2. The method of claim 1 wherein the step (3) is washing with a phosphate buffer to remove the viruses, to obtain the activated immunocytes.

3. The method of claim 1 wherein the body fluid containing the immunocytes is peripheral blood or malignant pleural effusion.

4. The method of claim 1 wherein the viruses are recombinant type herpes simplex viruses or wild-type herpes simplex viruses.

5. The method of claim 4 wherein the viruses are herpes simplex viruses type 1 or herpes simplex viruses type II.

6. The method of claim 1 wherein the viruses are recombinant herpes simplex viruses type II, with a preservation number of CGMCC No. 3600.

7. A method for treating tumor, comprising administering an effective amount of activated immunocytes, which is made by the method of claim 1.

8. The method of claim 7, wherein the effective part of the activated immunocytes is Peripheral Blood Mononuclear Cell.

9. The method of claim 7, wherein the effective part of the activated immunocytes is Natural Killer Cell.

10. The method of claim 7, wherein the effective amount of activated immunocytes is re-infused into the patient.
